(19) United States
(12) Patent Application Publication
(10) Pub. No.: US 2026/0327172 A1
(43) Pub. Date: Oct. 22, 2026

(54) SORBITOL DERIVATIVES AND METHODS THEREOF

(71) Applicants: Massachusetts Institute of Technology, Cambridge, MA (US); THE BOARD OF TRUSTEES OF THE UNIVERSITY OF ILLINOIS, URBANA, IL (US)

(72) Inventors: Scott E. Denmark, Urbana, IL (US); Tapas Adak, Urbana, IL (US); Travis Menard, Urbana, IL (US); Matthew Albritton, Urbana, IL (US); Federico Florit, Cambridge, MA (US); Martin D. Burke, Urbana, IL (US); Klavs F. Jensen, Cambridge, MA (US)

(21) Appl. No.: 19/427,259

(22) Filed: Dec. 19, 2025

Related U.S. Application Data
(60) Provisional application No. 63/737,683, filed on Dec. 21, 2024.

(51) Int. Cl. C07C 33/025; C07C 29/17; C07C 31/26; C07C 59/42; C07C 235/28

(52) U.S. Cl. CPC C07C 33/025; C07C 29/172; C07C 31/26; C07C 59/42; C07C 235/28

(57) ABSTRACT

This present disclosure is directed to compositions comprising allyl sorbitols and related derivatives, and an indium metal and/or indium metal, and methods (including continuous methods) of making such compounds.

### CROSS REFERENCE TO RELATED APPLICATIONS

This application claims priority to U.S. Application No. 63/737,683, filed on Dec. 21, 2024, the contents of which are hereby incorporated by reference in its entirety.

### STATEMENT OF FEDERALLY SPONSORED RESEARCH AND DEVELOPMENT

This invention was made with government support under 2019897, awarded by the National Science Foundation. The government has certain rights in the invention.

### FIELD OF THE INVENTION

The field of the invention relates generally to sorbitols, sorbitol derivatives and methods of making the same, more particularly to allyl sorbitols and allyl sorbitol derivatives and methods of making the same, and even more particularly to methods of making sorbitols in the presence of an indium salt and/or indium metal.

### BACKGROUND

This background information is provided for the purpose of making information believed by the applicant to be of possible relevance to the present invention. No admission is necessarily intended, nor should it be construed, that any of the information disclosed herein constitutes prior art against the present invention.

Carbohydrates are an abundant, inexpensive, and renewable biomass feedstock that could be a cornerstone for sustainable chemical manufacturing, but scalable and environmentally friendly methods that leverage these feedstocks are lacking. For example, 1-allyl sorbitol is the foundational building block for the polypropylene clarifying agent Millad® NX® 8000 which is produced on multi-ton scale annually, but the current manufacturing process requires super stoichiometric amounts of tin. A high-yielding allylation of unprotected carbohydrates in water using a catalytic amount of indium metal and either allylboronic acid or the pinacol ester (allylBpin) as donors is disclosed herein. Aldohexoses, aminohexoses, ketohexoses, and aldopentoses are all allylated in high yield under mild conditions, and the indium metal is recoverable and reusable with no loss of catalytic activity. Leveraging these features, this process was translated to a scalable continuous synthesis of 1-allyl sorbitol in flow with high yield and productivity through Bayesian optimization of reaction parameters.

### BRIEF DESCRIPTION OF THE FIGURES

The patent or application file contains at least one drawing executed in color. Copies of this patent or patent application publication with color drawing(s) will be provided by the Office upon request and payment of the necessary fee. The following drawings form part of the present specification and are included to further demonstrate certain aspects of the present invention. The invention may be better understood by reference to one or more of these drawings in combination with the detailed description of specific embodiments presented herein.

FIGS. 1A-1C. FIG. 1 displays schemes showing various methods for allylation of carbohydrates. FIG. 1A displays the synthetic route taken by Whitesides, who first demonstrated tin-mediated allylation of carbohydrates. FIG. 1B displays the synthetic route taken by Milliken, who produced a fourth generation clarifier, Millad® NX® 8000, which utilizes Whitesides' method to meet global demand of clarified PP. FIG. 1C displays the catalytic synthetic route disclosed herein for allylation of a sugar in an aqueous medium to address the existing limitations of the Millad® NX® 8000 synthesis.

FIGS. 2A-2B. FIG. 2 displays graphs and data showing the optimization of the allylation of glucose. FIG. 2A displays reaction profiles with insoluble indium catalysts at 2M ethanol/water (1:1). FIG. 2B displays reaction profiles with different solvents. FIG. 2C displays the optimum reaction conditions obtained by DOE. The indium powder was easily recovered as indium foil.

FIGS. 3A-3B. FIG. 3 displays the recoverability and reusability studies of In(0). FIG. 3A displays a 10 mmol scale allylation of D-arabinose with recovery of In(0) catalyst. FIG. 3B displays a reusability study detailing the 1.0 mmol scale allylation of D-ribose, repeated five times with the same piece of indium foil. Yield adjusted for residual water.

FIGS. 4A-4I. FIG. 4 displays the Bayesian optimization in the flow reactor. FIG. 4A displays the continuous packed-bed reactor set-up. FIGS. 4B-4E displays plots of measured and predicted yields for varying flowrate (FIG. 4B), temperature (FIG. 4C), allylBpin equivalence ration (FIG. 4D), and ethanol fraction (FIG. 4E). Black dots represent experimental measurements, and colored background dots show Gaussian Process (GP) predicted mean yields colored by their standard deviation shown in the scale bar. FIG. 4F displays Pareto front (blue line) between yield and productivity evaluated from the GP, demonstrating a trade-off between the competing objectives; the proposed process is always superior to the tin-mediated process (>60% syn isomer yield) and achieves good productivity at lab-scale; the black dots (initialization runs) and the red squares (optimization runs) demonstrate the efficient, explorative nature of the BO method used. FIGS. 4G-4I display productivity isocurves (colored by productivity) and yield derivation from optimum conditions (dotted black curves) showing that deviations from the Pareto-optimal conditions in flowrate and temperature (FIG. 4G), allylBpin (FIG. 4H), and ethanol (FIG. 4I) do not markedly affect productivity and yield.

FIG. 5. FIG. 5 displays a schematic of the flow setup for the preliminary experiments.

FIG. 6. FIG. 6 displays a graph showing the yield of syn isomer obtained in the preliminary experiments using different mass ratios between 100 mesh In(0) powder, and 100 μm diameter glass beads.

FIG. 7. FIG. 7 displays a scheme of the flow system for Bayesian optimization. TC=Temperature Controller, TE=Temperature Element and TI=Temperature Indicator.

FIG. 8. FIG. 8 displays a photo of the flow apparatus used for Bayesian optimization of yield and productivity.

FIG. 9. FIG. 9 displays an alternate scheme of the flow system. TC=Temperature Controller, TE=Temperature Element and TI=Temperature Indicator.

Particular non-limiting embodiments of the present invention will now be described with reference to accompanying drawings.

### DESCRIPTION

The industrial scale utilization of sustainable carbohydrate feedstocks is hindered by limited compatibility with traditional organic reactions and media. For example, the carbonyl group of reducing sugars is unavailable for most carbonyl functionalization reactions due to low equilibrium concentrations of “open chain” forms and low solubility in organic solvents. Additionally, the incompatibility of organometallic reagents towards moisture and unprotected hydroxyl groups results in competitive protonolysis and hinders C—C bond-forming reactions. Stereochemically and functionally rich carbohydrates have found widespread application in complex molecule synthesis pioneered by Hanessian using the “chiron” approach. Despite these elegant contributions, the use and modification of carbohydrates generally requires protecting group strategies to overcome these challenges. This reduces atom and step economy, increasing waste and cost. Robust, scalable, and sustainable methods for direct functionalization of unprotected carbohydrates and their derivatives in water are notable goals.

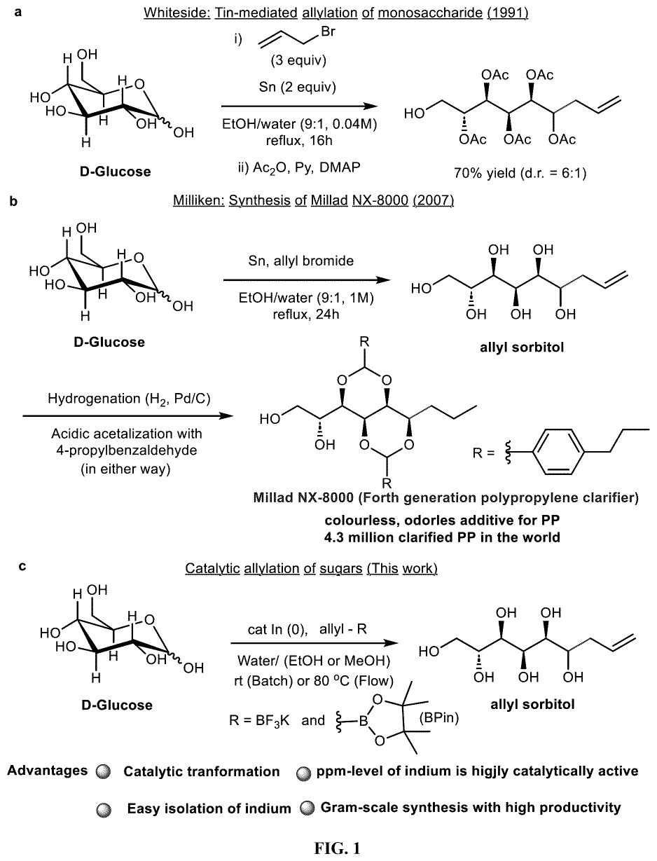

The metal-mediated Barbier-type allylation of unprotected sugars in aqueous media developed by Whitesides, Li, and Chan is the most notable illustration (FIG. 1). This advance was enabled by discoveries by Luche and Nokami-hydrolytically stable organometallic species could be prepared from the reaction of allyl halides and stoichiometric amounts of certain metals (Sn, Al, In, and Zn). These reagents react with aldehydes and ketones to furnish homoallylic alcohols and their extension to carbohydrates has emerged as a powerful synthetic process. However, the formation of stoichiometric amounts of metallic byproducts makes these reagents undesirable, particularly on a manufacturing scale. Moreover, these reagents exhibit poor reactivity with amino sugars, a biologically relevant class of carbohydrates.

Despite these limitations, Whitesides' Barbier-type allylation features prominently as the primary carbon-carbon bond forming reaction in the industrial synthesis of the most widely used polypropylene (PP) clarifying agent, Millad® NX® 8000. The market volume of PP in 2022 was approximately 79.01 million metric tons (MMT) and demand is projected to increase nearly 33% to 105 MMT by 2030. The benefits of clarified PP over other polymers, especially in terms of cost and sustainability, are likely to continue driving increasing demand for clarified polypropylene in turn creating need for greater quantities of such clarifying agents.

The current industrial synthetic process begins with a superstoichiometric tin-mediated allylation of glucose in ethanol/water using allyl bromide, a corrosive, volatile, and hazardous compound (FIG. 1). This reaction produces superstoichiometric amounts of tin byproducts that should be neutralized and separated from the reaction mixture, ultimately increasing the complexity of the process and the overall cost. Obviously, development of a new allylation method for glucose and other unprotected sugars in a protic medium on a process scale is desirable. The inventors surprisingly discovered that an aqueous phase, indium-catalyzed allylation of glucose and other carbohydrates that requires only catalytic amounts of indium and utilizes pinacol allylboronic ester (allylBpin) as the three-unit carbon source can be made both in batch and in flow, with the latter optimized for scalability using machine learning.

One aspect of the invention pertains to a composition comprising one or more compounds chosen from allyl sorbitol, allenyl sorbitol, propargyl sorbitol, propyl sorbitol, and ((R)-1-((2R,4R,4aS,6R,8R,8aS)-8-propyl-2,6-bis(4-propylphenyl)tetrahydro-[1,3]dioxino[5,4-d][1,3]dioxin-4-yl)ethane-1,2-diol). In some embodiments, the composition further comprises indium and/or an indium salt. In other embodiments, said indium salt is chosen from InCl, InI, InBr, InCl₃, InBr₃, InI₃, In(OAc)₃, In(OTf)₃In₂O₃, or In(OH)₃, and combinations thereof.

Another aspect of the invention pertains to a method of preparing a compound of Formula I, said method comprising reacting a monosaccharide and a compound of any of Formulas II, IIA, or IIB in the presence of indium metal and/or an indium salt and at least one solvent, wherein A may be chosen from —CH₂—,

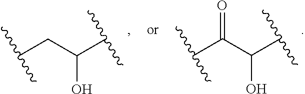 [SMILES: *CC(*)O ; *C(=O)C(*)O]

### R¹ may be H, —OH, or

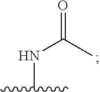 [SMILES: *NC(C)=O]

R² may be H or —CH₂OH; R³ and R⁴ may be independently chosen from hydroxy, halogen, and C₁₋₆ alkoxy (e.g., methoxy, ethoxy, propoxy, butoxy, isopropoxy, sec-butoxy, tert-butoxy, CH₂-tert-butoxy, etc.) or combinations thereof; or R³ and R⁴, taken together with the B atom to which they may be attached, form a 5-membered, a 6-membered, or a 7-membered heterocyclic ring system; and Z may be

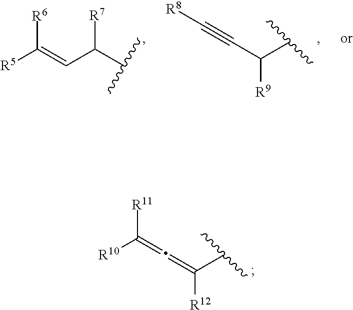

wherein R⁵-R¹² may be independently chosen from H, hydroxy, halogen, and C₁₋₆ alkyl (e.g., methyl, ethyl, propyl, butyl, isopropyl, sec-butyl, tert-butyl, CH₂-tert-butyl, etc.) or a combination thereof.

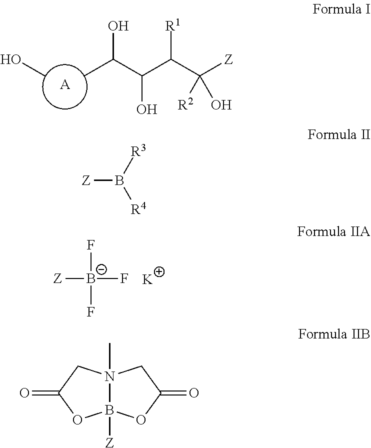

In some embodiments, said monosaccharide is a hexose or a pentose. In other embodiments, said monosaccharide is glucose, or D-glucose. In other embodiments, said indium salt is chosen from InCl, InI, InBr, InCl₃, InBr₃, InI₃, In(OAc)₃, In(OTf)₃In₂O₃, or In(OH)₃, and combinations thereof.

A further aspect of the invention pertains to a continuous process of preparing a compound of Formula I comprising reacting a monosaccharide and a compound of any of Formulae II, IIA, or IIB in the presence of indium metal and/or an indium salt and at least one solvent; wherein A may be chosen from CH₂,

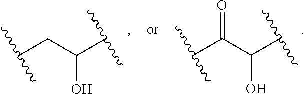 [SMILES: *CC(*)O ; *C(=O)C(*)O]

### R¹ may be H, —OH, or

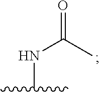 [SMILES: *NC(C)=O]

R² may be H or —CH₂OH; R³ and R⁴ may be independently chosen from H, hydroxy, halogen, and C₁₋₆ alkoxy (e.g., methoxy, ethoxy, propoxy, butoxy, isopropoxy, sec-butoxy, tert-butoxy, CH₂-tert-butoxy, etc.) or combinations thereof; or R³ and R⁴, taken together with the B atom to which they may be attached to form a 5-membered, a 6-membered, or a 7-membered heterocyclic ring system; and Z may be

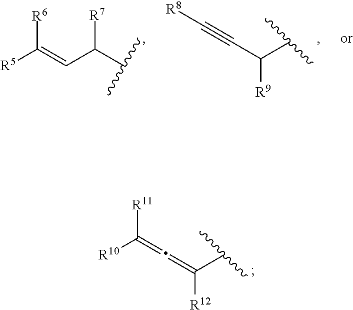

wherein R⁵-R¹² may be independently chosen from H, hydroxy, halogen, and C₁₋₆ alkyl (e.g., methyl, ethyl, propyl, butyl, isopropyl, sec-butyl, tert-butyl, CH₂-tert-butyl, etc.) or combinations thereof. In some embodiments, said reactants and solvent are introduced into a reactor via separate streams. Said reactor may be a continuous reactor such as a continuous flow tubular reactor.

In other embodiments, said indium metal and/or said indium salt is arranged to form a catalytic bed. Said catalytic bed may be a bed reactor, such as a fluid bed reactor, a fixed bed reactor, or may be a reactor with a catalyst supported on an inert material. In other embodiments, the said reaction may be optionally heated. The reaction may be heated at a temperature in the range of about 20° C. or about room temperature, to about 100° C., or in the range of about 50° C. to about 100° C., or the reaction may be heated to a temperature of about 80° C. In further embodiments, said reactants and said solvent are optionally introduced into the reactor at a flow rate in the range of about 15 μL/min to about 55 μL/min, or about 15 μL/min to about 20 μL/min, or about 17.5 μL/min, or about 45 μL/min to about 55 μL/min, or about 52.5 μL/min. In some embodiments, said reactants have a contact time with said reactor of up to 30 minutes. In other embodiments, said indium metal and/or an indium salt may be reused for more than one reaction. In further embodiments, said indium metal and/or said indium salt is mixed with an inert material such as glass beads, ceramic beads, or metallic beads.

In some embodiments, Formula IA and/or Formula IB or stereoisomers thereof may be prepared by the synthetic route outlined in Scheme 1. The reaction disclosed in Scheme 1 may be modified using known methods to prepare compounds of Formula IA and/or Formula IB. In some embodiments, Formula IA and Formula IB may be prepared by a method comprising reacting a monosaccharide of Formula III (and/or stereoisomers thereof) and a compound of Formula IIC, in the presence of indium metal and/or an indium salt such as indium halide (e.g. InBr₃) to obtain Formula IA and/or IB. Compounds of Formula IA and/or IB may be subject to hydrogenation, for example by reacting further compounds of Formula IA and/or IB with hydrogen (H₂) in the presence of a catalyst such as a platinum (Pt) catalyst to obtain propyl sorbitol (e.g., a stereoisomer of Formula IV). The methods in Scheme 1 may be applied.

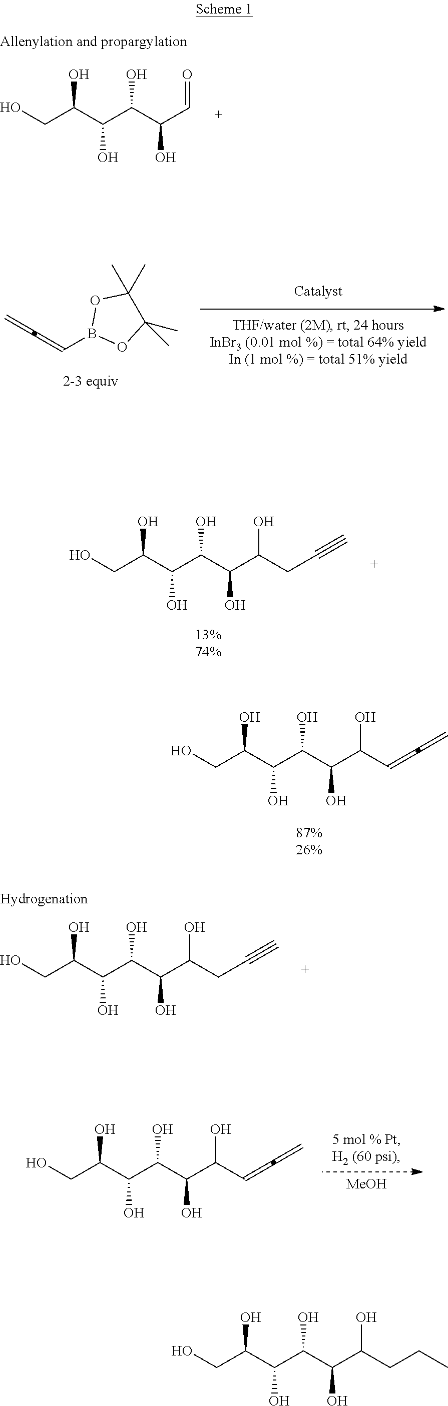

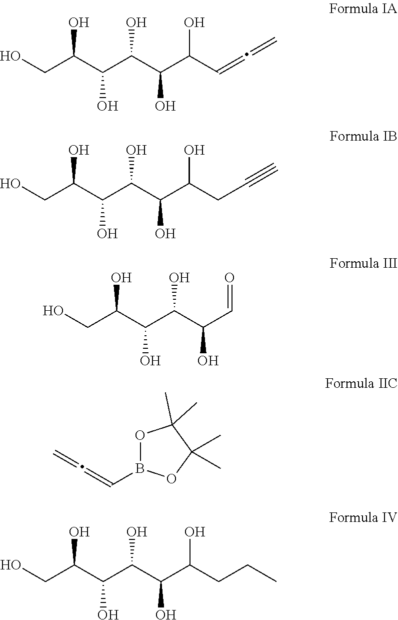

One aspect of the invention pertains to a compound of Formula I, wherein said compound is prepared according to any of the methods previously mentioned.

All publications mentioned herein are incorporated by reference to the extent they support the present invention. Further aspects of the invention are described in the following: Nature 640, 94-99 (2025) and “Highly Catalytic Allylation of Native Carbohydrates in Water with Indium Mechanists and Spectroscopic Studies” Albritton et al., submitted to the Journal of the American Chemical Society, expected publication 2026, both of which are hereby incorporated by reference in their entirety.

### Definitions

For the purposes of promoting an understanding of the principles of the invention, reference will now be made to certain embodiments and specific language will be used to describe the same. It will nevertheless be understood that no limitation of the scope of the invention is thereby intended, and alterations and modifications in the illustrated invention, and further applications of the principles of the invention as illustrated therein are herein contemplated as would normally occur to one skilled in the art to which the invention relates.

Unless defined otherwise, all technical and scientific terms used herein have the same meaning as commonly understood by one of ordinary skill in the art to which this invention pertains.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used).

The use of “or” means “and/or” unless stated otherwise.

The use of “a” or “an” herein means “one or more” unless stated otherwise or where the use of “one or more” is clearly inappropriate.

The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” refers to a ±10% variation from the nominal value. It is to be understood that such a variation is always included in any given value provided herein, whether or not it is specifically referred to.

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Moreover, all ranges disclosed herein are to be understood to encompass any and all subranges (including all fractional and whole values) subsumed therein.

The term “OTf” refers to the triflate anion, [CF₃SO₃]⁻

The term “rt” refers to room temperature.

The term “alkyl” as used herein by itself or as part of another group refers to both straight and branched chain radicals, and cyclic alkyl groups. In one embodiment, the alkyl group has 1-12 carbons. In another embodiment, the alkyl group has 1-7 carbons. In another embodiment, the alkyl group has 1-6 carbons. In another embodiment, the alkyl group has 1-4 carbons. The term “alkyl” may include methyl, ethyl, propyl, isopropyl, butyl, s-butyl, t-butyl, isobutyl, pentyl, hexyl, isohexyl, heptyl, 4,4-dimethylpentyl, octyl, 2,2,4-trimethylpentyl, nonyl, decyl, undecyl, and dodecyl.

The term “heteroalkyl,” by itself or in combination with another term, means, unless otherwise stated, a linear or branched chain having at least one carbon atom and at least one heteroatom selected from the group consisting of O, N, S, P, and Si. In certain embodiments, the heteroatoms are selected from the group consisting of O, and N. The heteroatom(s) may be placed at any interior position of the heteroalkyl group or at the position at which the alkyl group is attached to the remainder of the molecule. Up to two heteroatoms may be consecutive.

The term “alkylene” as used herein refers to straight and branched chain alkyl linking groups, i.e., an alkyl group that links one group to another group in a molecule. In some embodiments, the term “alkylene” may include —(CH₂)ₙ— where n is 2-8.

The term “aryl” as used herein refers to a polyunsaturated hydrocarbon substituent. Aryl groups can be monocyclic or polycyclic (e.g., 2 to 3 rings that are fused together or linked covalently). Non-limiting examples of aryl and heteroaryl rings are phenyl, naphthyl, pyranyl, pyrrolyl, pyrazinyl, pyrimidinyl, pyrazolyl, pyridinyl, furanyl, thiophenyl, thiazolyl, imidazolyl, isoxazolyl, and the like. Aryl groups can be substituted or unsubstituted.

The term “heteroaryl” as used herein refers to groups having 5 to 14 ring atoms; 6, 10 or 14 π-electrons shared in a cyclic array; and containing carbon atoms and 1, 2 or 3 oxygen, nitrogen or sulfur heteroatoms. A heteroaryl group can be attached to the remainder of the molecule through a carbon or heteroatom. Especially preferred heteroaryl groups include 1,2,3-triazole, 1,2,4-triazole, 5-amino 1,2,4-triazole, imidazole, oxazole, isoxazole, 1,2,3-oxadiazole, 1,2,4-oxadiazole, 3-amino-1,2,4-oxadiazole, 1,2,5-oxadiazole, 1,3,4-oxadiazole, pyridine, 2-aminopyridine, 4-aminopyridine, 2-aminoimidazoline, and 4-aminoimidazoline.

An “amino” group refers to an —NH₂ group.

An “amido” group refers to an —CONH₂ group. An alkylamido group refers to an —CONHR group wherein R is as defined above. A dialkylamido group refers to an —CONRR′ group wherein R and R′ are as defined above.

The term “halogen” or “halo” as used herein by itself or as part of another group refers to chlorine, bromine, fluorine or iodine.

The term “hydroxy” or “hydroxyl” as used herein by itself or as part of another group refers to an —OH group.

An “alkoxy” group refers to an —O-alkyl group wherein “alkyl” is as defined above. In one embodiment, the alkyl group has 1-12 carbons. In another embodiment, the alkyl group has 1-7 carbons. In a further embodiment, the alkyl group has 1-6 carbons. In another embodiment, the alkyl group has 1-4 carbons.

The term “heterocycle” or “heterocyclic ring”, as used herein except where noted, represents a stable 5- to 7-membered monocyclic-, or stable 7- to 11-membered bicyclic heterocyclic ring system, any ring of which may be saturated or unsaturated, and which consists of carbon atoms and from one to three heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. Rings may contain one oxygen or sulfur, one to three nitrogen atoms, or one oxygen or sulfur combined with one or two nitrogen atoms. The heterocyclic ring may be attached at any heteroatom or carbon atom that results in the creation of a stable structure.

The term “alkylamino” as used herein by itself or as part of another group refers to an amino group which is substituted with one alkyl group having from 1 to 6 carbon atoms. The term “dialkylamino” as used herein by itself or as part of another group refers to an amino group which is substituted with two alkyl groups, each having from 1 to 6 carbon atoms.

The term “arylamine” or “arylamino” as used herein by itself or as part of another group refers to an amino group which is substituted with an aryl group, as defined above.

As used herein, the term “arylalkyl” denotes an alkyl group substituted with an aryl group, for example, Ph-CH₂— etc. In certain embodiments, the term “cycloalkyl” includes bicyclic ring systems. The bicyclic ring system may be in the form of a bridged, fused, or spiro form.

Various groups are described herein as substituted or unsubstituted (i.e., optionally substituted). Optionally substituted groups may include one or more substituents independently selected from: halogen, nitro, cyano, hydroxy, amino, mercapto, formyl, carboxy, oxo, carbamoyl, alkyl, heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl, substituted or unsubstituted aryl, and substituted or unsubstituted heteroaryl. In certain aspects the optional substituents may be further substituted with one or more substituents independently selected from: halogen, nitro, cyano, hydroxy, amino, mercapto, formyl, carboxy, carbamoyl (—C(O)NR₂), unsubstituted alkyl, unsubstituted heteroalkyl, alkoxy, alkylthio, alkylamino, (alkyl)₂amino, alkylsulfinyl, alkyl sulfonyl, aryl sulfonyl, unsubstituted cycloalkyl, unsubstituted heterocyclyl, unsubstituted aryl, or unsubstituted heteroaryl. Exemplary optional substituents include, but are not limited to: —OH, oxo (═O), —Cl, —F, Br, C₁₋₄alkyl, phenyl, benzyl, —NH₂, —NH(C₁₋₄alkyl), —N(C₁₋₄alkyl)₂, —NO₂, —S(C₁₋₄alkyl), —SO₂(C₁₋₄alkyl), —CO₂(C₁₋₄alkyl), and —O(C₁₋₄alkyl).

As used herein, the term “allyl sorbitol” refers to any stereoisomer of non-8-ene,1,2,3,4,5,6-hexaol. In some embodiments, “allyl sorbitol” may refer to the molecule with the IUPAC name ((2R,3R,4R,5S,6R)-non-8-ene-1,2,3,4,5,6-hexaol).

As used herein, the term “allenyl sorbitol” refers to any stereoisomer of nona-7,8-diene-1,2,3,4,5,6-hexaol. In some embodiments, “allenyl sorbitol” may refer to the molecule with the IUPAC name ((2R,3R,4R,5S,6R)-nona-7,8-diene-1,2,3,4,5,6-hexaol).

As used herein, the term “propargyl sorbitol” refers to any stereoisomer of non-8-yne-1,2,3,4,5,6-hexaol. In some embodiments, “propargyl sorbitol” refers to the molecule with the IUPAC name ((2R,3R,4R,5S,6R)-non-8-yne-1,2,3,4,5,6-hexaol).

As used herein, the term “propyl sorbitol” refers to any stereoisomer of nonane-1,2,3,4,5,6-hexaol. In some embodiments, “propyl sorbitol” refers to the molecule with the IUPAC name ((2R,3R,4R,5S,6R)-nonane-1,2,3,4,5,6-hexaol).

As used herein, the term “MILLAD® NX® 8000” refers to the molecule with the IUPAC name ((R)-1-((2R,4R,4aS,6R,8R,8aS)-8-propyl-2,6-bis(4-propylphenyl)tetrahydro-[1,3]dioxino[5,4-d][1,3]dioxin-4-yl)ethane-1,2-diol).

According to the present disclosure the term “continuous method”, as used herein, refers to synthetic methods in which various streams of reagents are continuously introduced by pumps, syringes, or other means known within the art to a reactor wherein a reaction takes place. At the same time, an outlet stream (effluent) is collected from the reactor. The reactor operates over time without relevant changes of the effluent composition after an initial transient, up to steady state. It is opposed by “batch” or “fed-batch” methods. Further, a continuous method is a production method in which raw materials are continuously fed into a process and transformed into intermediate or final materials in an uninterrupted flow. This method enables steady-state operation, precise control of processing parameters (such as temperature, composition, and flow rate), and real-time monitoring to ensure uniform material properties and consistent product quality.

As used herein, the term “contact time” refers to the time that a reactant in a continuous reactor system spends in direct contact with other reactants, solvent and/or indium/indium salt within the reactor. It may be adjusted using known methods based on the volume of the reactor and the flow rate of the reactants.

It is to be understood that both the foregoing descriptions are exemplary, and thus do not restrict the scope of the invention.

### LIST OF EMBODIMENTS

The following is a list of non-limiting embodiments:
1. A composition comprising one or more compounds chosen from allyl sorbitol, allenyl sorbitol, propargyl sorbitol, propyl sorbitol, and ((R)-1-((2R,4R,4aS,6R,8R,8aS)-8-propyl-2,6-bis(4-propylphenyl)tetrahydro-[1,3]dioxino[5,4-d][1,3]dioxin-4-yl)ethane-1,2-diol).
2. The composition of embodiment 1, wherein said composition further comprises indium metal and/or an indium salt.
3. The composition of any of the preceding embodiments, wherein said indium salt is chosen from InCl, InI, InBr, InCl₃, InBr₃, InI₃, In(OAc)₃, In(OTf)₃In₂O₃, or In(OH)₃, and combinations thereof.
4. The composition of any of the preceding embodiments, wherein said indium salt is In(OTf)₃.
5. The composition of any of the preceding embodiments, wherein said composition comprises allyl sorbitol, and indium metal and/or an indium salt.
6. The composition of any of the preceding embodiments, wherein, said allyl sorbitol is chosen from

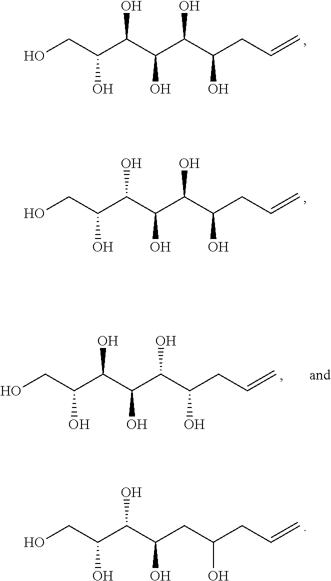

7. The composition of any of the preceding embodiments, wherein the composition comprises indium metal and/or an indium salt and any of the following compounds:

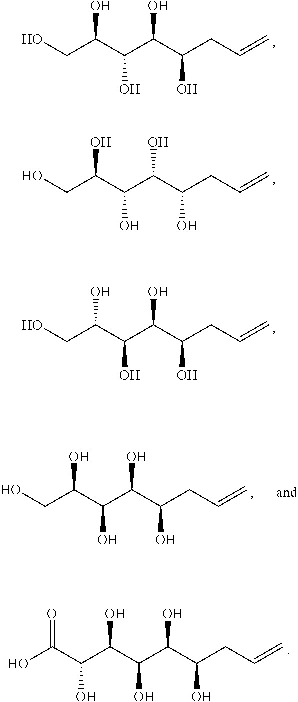 [SMILES: C=CC[C@@H](O)[C@H](O)[C@H](O)[C@H](O)CO ; C=CC[C@H](O)[C@@H](O)[C@H](O)[C@H](O)CO ; C=CC[C@@H](O)[C@H](O)[C@@H](O)[C@@H](O)CO ; C=CC[C@@H](O)[C@H](O)[C@@H](O)[C@H](O)CO ; C=CC[C@@H](O)[C@H](O)[C@@H](O)[C@H](O)[C@H](O)C(=O)O]

8. The composition of any of the preceding embodiments, wherein said composition comprises propyl sorbitol.
9. The composition of any of the preceding embodiments, wherein said propyl sorbitol is either of:

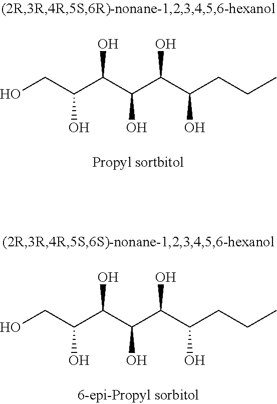

10. The composition of any of the preceding embodiments, wherein said indium metal and/or indium salt is present at a concentration of at least 16 ppm, or about 16 ppm to about 300 ppm, or about 30 ppm to about 150 ppm, or about 70 ppm to about 100 ppm.
11. The composition of any of the preceding embodiments, wherein said indium metal and/or indium salt is present at a concentration of at least 16 ppm.
12. The composition of any of the preceding embodiments, wherein said composition does not include (or is free of) compounds of Formulae II, IIA, and/or IIB:

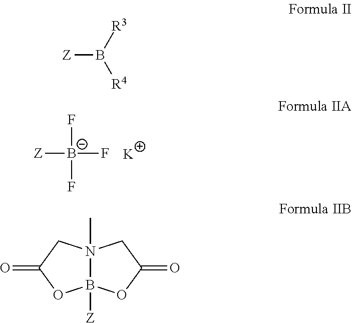

wherein R³ and R⁴ are independently chosen from H, hydroxy, halogen, and C₁₋₆ alkyl or combinations thereof;
or R³ and R⁴, taken together with the B atom to which they are attached, form a 5-membered, a 6-membered, or a 7-membered heterocyclic ring system; and
   Z is

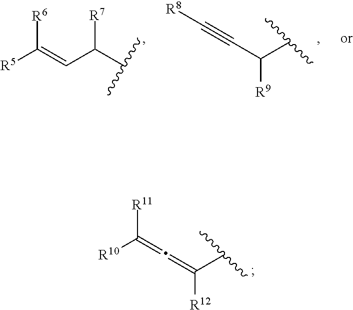

and
wherein R⁵-R¹² are independently chosen from H, hydroxy, halogen, and C₁₋₆ alkyl or a combination thereof.
13. The composition of any of the preceding embodiments, wherein said composition does not include (or is free of) allyl boronic acid pinacol ester (Formula IID).

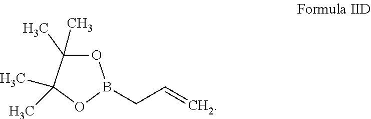

14. The composition of any of the preceding embodiments, wherein said composition does not include (or is free of) allylBF₃K.
15. The composition of any of the preceding embodiments, wherein said C₁₋₆ alkyl is chosen from methyl, ethyl, propyl, butyl, isopropyl, sec-butyl, tert-butyl, or CH₂-tert-butyl.
16. A method of preparing a compound of Formula I, said method comprising reacting a monosaccharide and a compound of any of Formulae II, IIA, or IIB in the presence of indium metal and/or an indium salt and at least one solvent

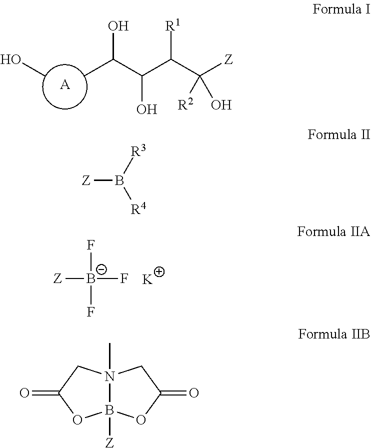

wherein:
   A is chosen from —CH₂—,

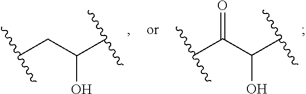 [SMILES: *CC(*)O ; *C(=O)C(*)O]

R¹ is H, —OH, or

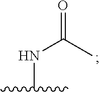 [SMILES: *NC(C)=O]

R² is H or —CH₂OH;
R³ and R⁴ are independently chosen from hydroxy, halogen, and C₁₋₆ alkoxy or combinations thereof; or R³ and R⁴, taken together with the B atom to which they are attached, form a 5-membered, a 6-membered, or a 7-membered heterocyclic ring system; and
Z is

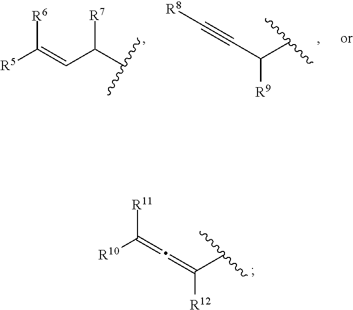

wherein R⁵-R¹² are independently chosen from H, hydroxy, halogen, and C₁₋₆ alkyl or a combination thereof.
17. The method of any of the preceding embodiments, wherein said monosaccharide is chosen from glucose, D-glucose, hexose, pentose, and combinations thereof.
18. The method of any of the preceding embodiments, wherein said monosaccharide is glucose.
19. The method of any of the preceding embodiments, wherein said compounds of Formula 1A and/or Formula IB are prepared by the synthetic route according to Scheme 1:

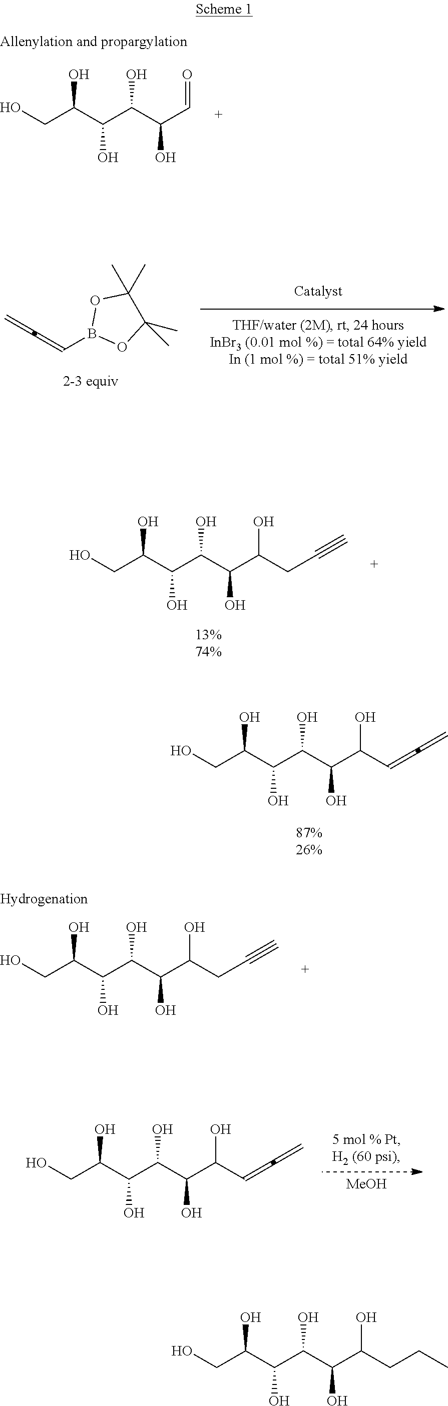

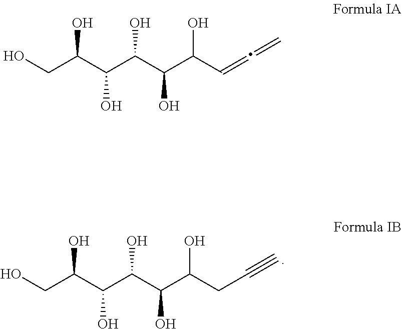

20. The method of any of the preceding embodiments, wherein said compounds of Formula IA and/or Formula IB are prepared by reacting a monosaccharide of Formula III and/or stereoisomers thereof, and a compound of Formula IIC, in the presence of indium metal and/or an indium salt

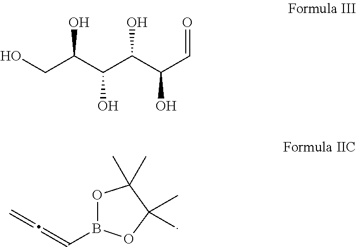

21. The method of any of the preceding embodiments, wherein said indium salt is an indium halide (e.g. InBr₃).
22. The method of any of the preceding embodiments, wherein said indium salt is InBr₃.
23. The method of any of the preceding embodiments, wherein said compounds of Formula IA and/or IB are reacted with hydrogen in the presence of a catalyst to obtain a compound of Formula IV, or a stereoisomer thereof:

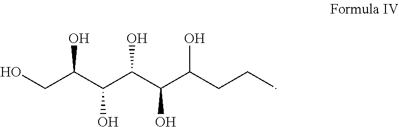

24. The method of any of the preceding embodiments, wherein said catalyst is platinum.
25. The method of embodiments 16-24, with the proviso that when said monosaccharide is glucose, then for Formula II, if Z is

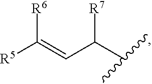

and R⁵, R⁶ and R⁷ are independently H, then R³ and R⁴ taken together is not:

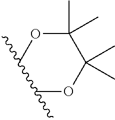

and does not form a compound of Formula IID:

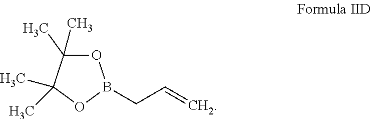

26. The method of embodiments 16-25, with the proviso that when said monosaccharide is glucose, then for Formula IIA, Z is not

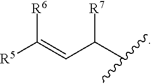

27. The method of embodiments 16-26, with the proviso that when said monosaccharide is glucose, then Formula IIA is not allylBF₃K.
28. The method of embodiments 16-27, with the proviso that when said monosaccharide is glucose, Formula II is not allyl boronic acid pinacol ester (Formula IID).

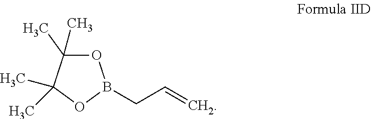

29. The method of any of the preceding embodiments, wherein said C₁₋₆ alkyl is chosen from methyl, ethyl, propyl, butyl, isopropyl, sec-butyl, tert-butyl, or CH₂-tert-butyl.
30. A continuous process of preparing a compound of Formula I, said method comprising reacting a monosaccharide (“reactant 1”) and a compound of any of Formulae II, IIA, or IIB (“reactant 2”) in the presence of indium metal and/or an indium salt and at least one solvent,

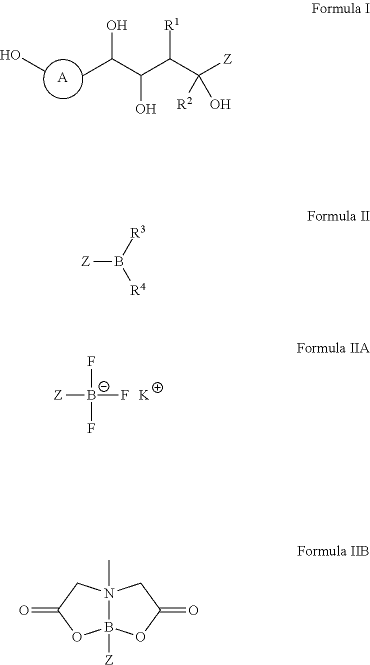

wherein:
   A is chosen from CH₂,

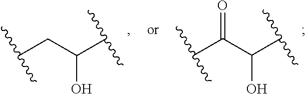 [SMILES: *CC(*)O ; *C(=O)C(*)O]

R¹ is H, —OH, or

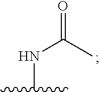 [SMILES: *NC(C)=O]

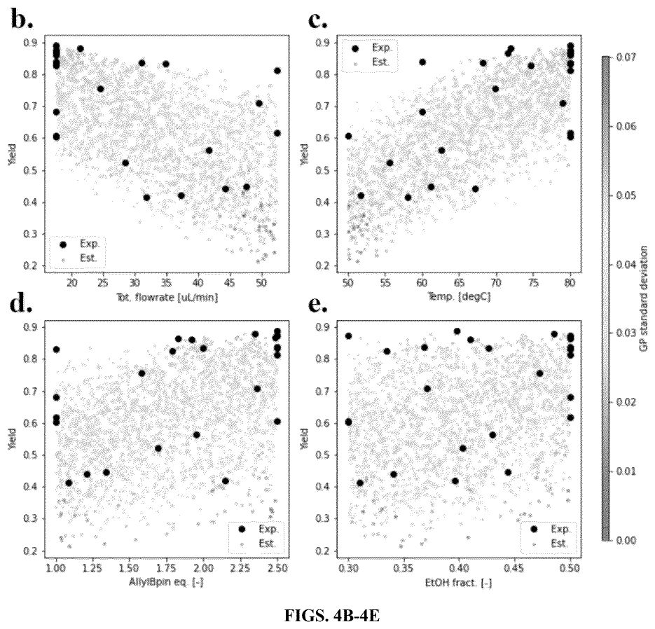

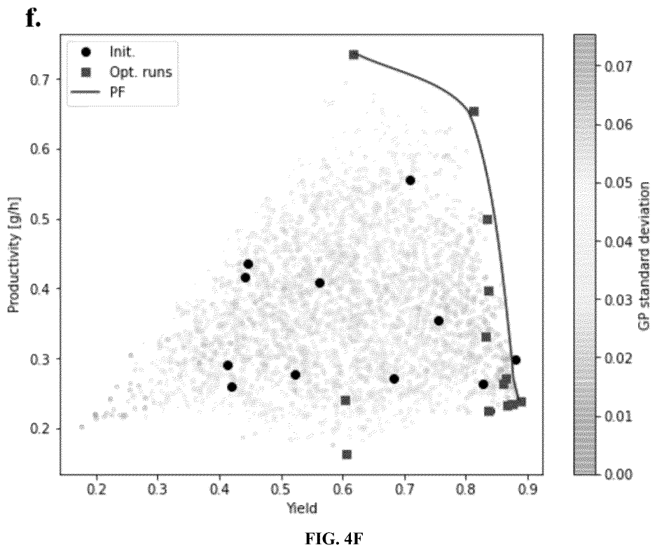

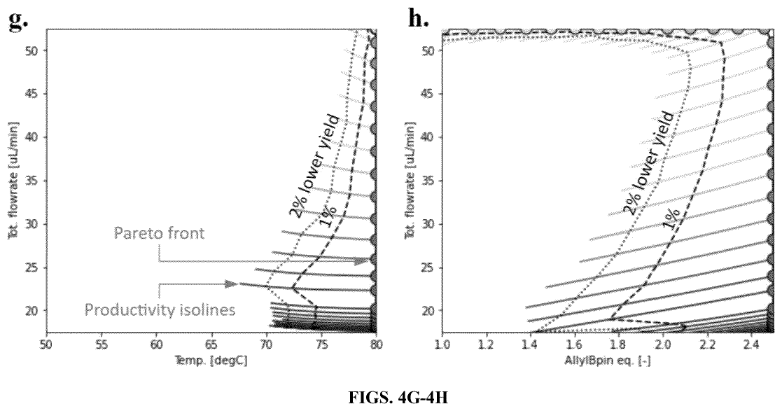

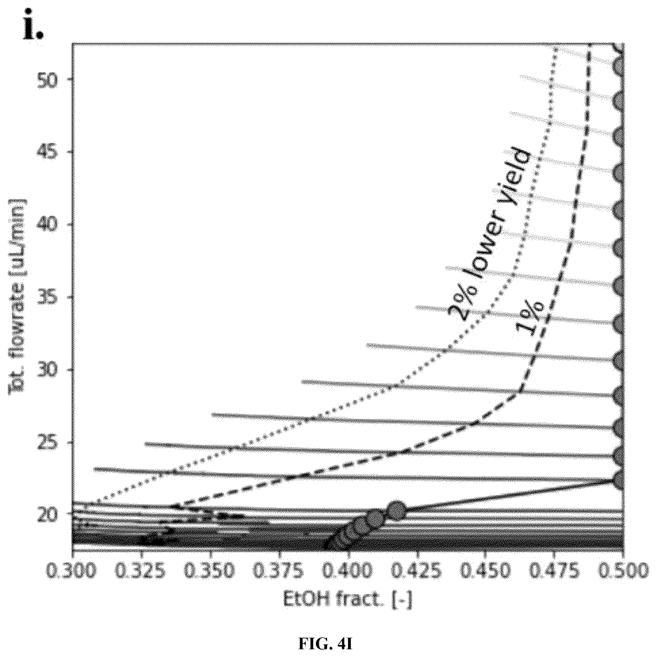

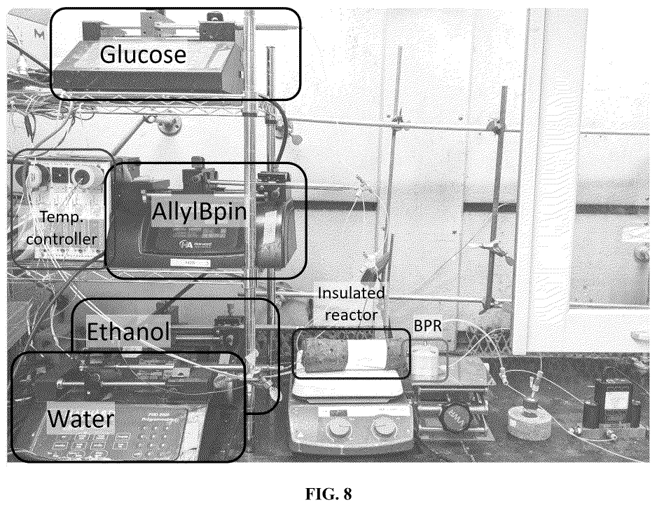

and
R² is H or —CH₂OH;
R³ and R⁴ are independently chosen from H, hydroxy, halogen, and C₁₋₆ alkoxy or combinations thereof; or
R³ and R⁴, taken together with the B atom to which they are attached, form a 5-membered, a 6-membered, or a 7-membered heterocyclic ring system; and
Z is

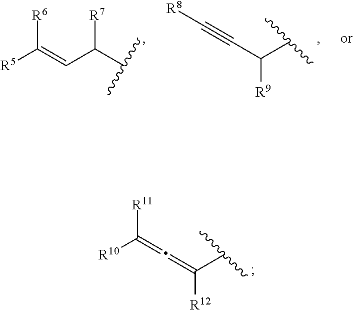

wherein R⁵-R¹² are independently chosen from H, hydroxy, halogen, and C₁₋₆ alkyl or combinations thereof,
wherein said reactants and solvent are introduced into a reactor via separate streams (see e.g., FIG. 4A and FIG. 8);
wherein said indium metal and/or an indium salt is arranged to form a catalytic bed,
optionally wherein said reaction is heated;
optionally wherein said indium metal and/or an indium salt is reused for more than one reaction;
optionally wherein said indium metal and/or an indium salt is mixed with an inert material such as glass beads, ceramic beads, or metallic beads.
31. The method of any of the preceding embodiments, wherein said reactants and solvent are independently introduced into the reactor at a flow rate in the range of about 15 μL/min to about 55 μL/min, or about 15 μL/min to about 20 μL/min, or about 17.5 μL/min, or about 45 μL/min to about 55 μL/min, or about 52.5 μL/min.
32. The method of any of the preceding embodiments, wherein said reactants have a contact time with the indium metal/indium salt and solvent within said reactor of about 30 minutes.
33. The method of any of the preceding embodiments, wherein said monosaccharide is a hexose and/or a pentose.
34. The method of any of the preceding embodiments, wherein said indium salt is chosen from InCl, InI, InBr, InCl₃, InBr₃, InI₃, In(OAc)₃, In(OTf)₃In₂O₃, or In(OH)₃, and combinations thereof.
35. The method of any of the preceding embodiments, wherein said reactor is a continuous reactor such as continuous flow tubular reactor.
36. The method of any of the preceding embodiments, wherein said catalytic bed is a bed reactor, such as fluid bed reactor, a fixed bed reactor, or a reactor with catalyst supported on an inert material.
37. The method of any of the preceding embodiments, wherein said reaction is heated to a temperature in the range of about 20° C. (e.g., room temperature) to about 100° C., or in the range of about 50° C. to about 100° C., or about 80° C.
38. The method of any of the preceding embodiments, wherein said monosaccharide is provided in a first steam, said compound of any of Formulae II, IIA, or IIB is provided in a second stream, said solvent is provided in one or more streams; wherein said indium metal and/or indium salt is provided in a reaction vessel comprising a packing material.
39. The method of any of the preceding embodiments, wherein said streams are premixed and/or form one stream before entering the reactor.
40. The method of any of the preceding embodiments, wherein each stream has a flow rate.
41. The method of any of the preceding embodiments, wherein said packing material comprises packed glass beads.
42. The method of any of the preceding embodiments, wherein said packed glass beads are 100 μm glass beads.
43. The method of any of the preceding embodiments, wherein said method comprises heating said reaction at a temperature, at a concentration/equivalence of said compound of Formula II, and at an ethanol fraction in the range of about 30% to about 50%, or about 40%.
44. The method of any of the preceding embodiments, wherein said temperature is in the range of about 50° C. to about 100° C., or is about 80° C.
45. The method of any of the preceding embodiments, wherein said equivalence is in the range of about 1 equivalence to about 2.5 equivalence of said compound.
46. The method of any of the preceding embodiments, wherein said compound of Formula I is one of:

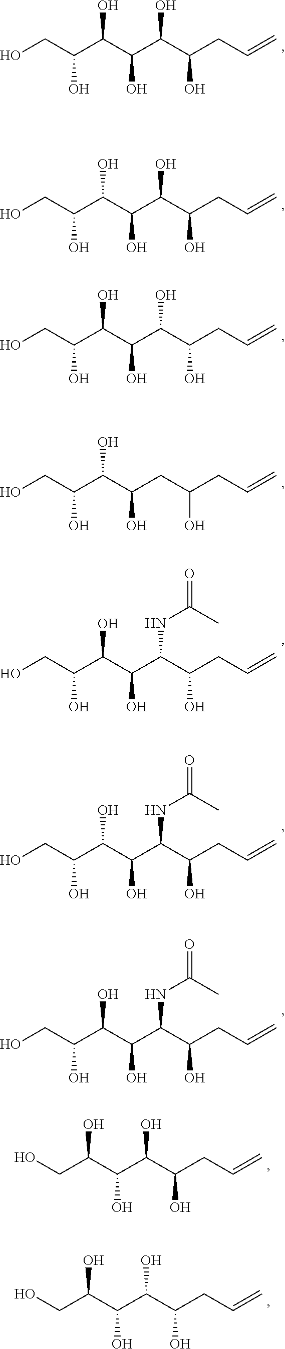

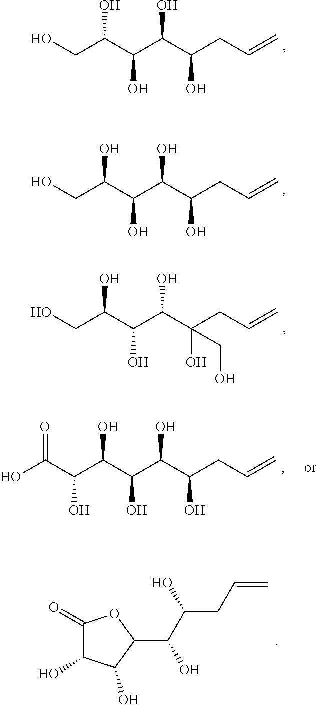 [SMILES: C=CC[C@@H](O)[C@H](O)[C@@H](O)[C@@H](O)CO ; C=CC[C@@H](O)[C@H](O)[C@@H](O)[C@H](O)CO ; C=CCC(O)(CO)[C@@H](O)[C@H](O)[C@H](O)CO ; C=CC[C@@H](O)[C@H](O)[C@@H](O)[C@H](O)[C@H](O)C(=O)O ; C=CC[C@@H](O)[C@H](O)C1OC(=O)[C@@H](O)[C@H]1O]

47. The method of any of the preceding embodiments, wherein said monosaccharide is chosen from D-glucose, D-galactose, D-mannose, 2-deoxy-D-galactose, N—Ac-D-mannosamine, N—Ac-D-galactosamine, N—Ac-D-glucosamine, D-ribose, D-arabinose, L-arabinose, D-xylose, D-fructose, D-glucuronolactone, and D-glucoronic acid.
48. The method of any of the preceding embodiments, wherein R³ and R⁴, taken together with the B atom to which they are attached, form a 5-membered, a 6-membered, or a 7-membered heterocyclic ring system with at least two oxygen or nitrogen atoms (e.g., two oxygen atoms connect directly to the boron atom).
49. The method of any of the preceding embodiments, wherein said 5-membered, 6-membered, or 7-membered heterocyclic ring system is any of:

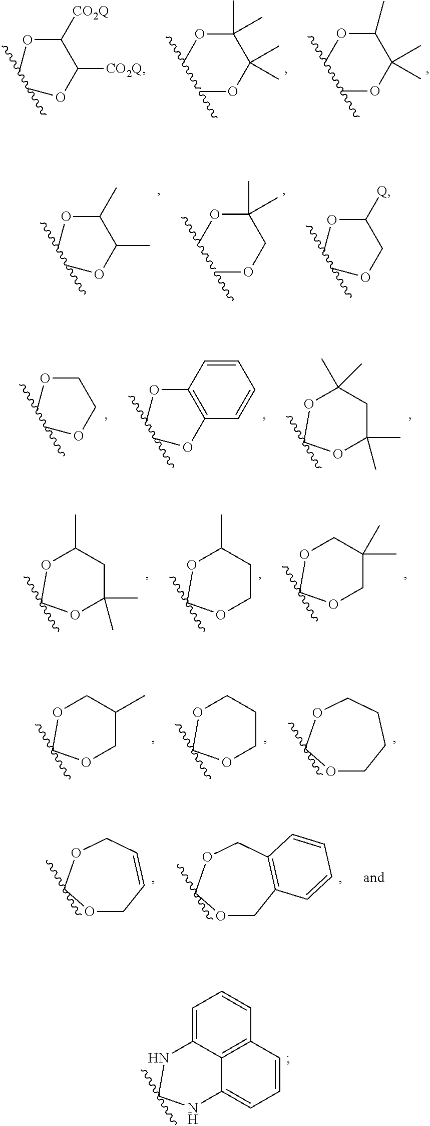

wherein Q is C₁₋₆ alkyl.
50. The method of any of the preceding embodiments, wherein said indium metal is in the form of a powder or foil.
51. The method of any of the preceding embodiments, wherein said powder has a mesh value in the range of about 60 to about 325, or mesh 325, or said mesh value is about 100.
52. The method of any of the preceding embodiments, wherein said indium salt is chosen from InCl₃, InBr₃, InI₃, In(OTf)₃, In(OAc)₃, InI, InBr, InCl, In₂O₃, or In(OH)₃, or combinations thereof.
53. The method of any of the preceding embodiments, wherein said solvent is chosen from water, methanol, ethanol, isopropanol, acetonitrile, tetrahydrofuran, and dimethylformamide, and or a combination thereof.
54. The method of any of the preceding embodiments, wherein said solvent is a mixture of water and ethanol.
55. The method of any of the preceding embodiments, wherein said mixture of water and ethanol is about 60% water and about 40% ethanol.
56. The continuous method of any of the preceding embodiments, wherein said C₁₋₆ alkyl is chosen from methyl, ethyl, propyl, butyl, isopropyl, sec-butyl, tert-butyl, or CH₂-tert-butyl.
57. A compound of Formula I, wherein said compound is prepared according to any of the preceding embodiments.

### EXAMPLES

The following examples are provided solely to illustrate the present invention and are not intended to limit the scope of the invention, described herein.

### Example 1. Reaction Optimization in Batch

Allylation of unprotected glucose under aqueous conditions as an efficient, high-yielding, and environmentally benign process for the synthesis of 1-allyl sorbitol, a building block of Millad© NX® 8000 was developed. Initial goals were to develop a process that avoids allyl halides, uses inexpensive feedstock starting materials, operates catalytically, and involves simple purification. Accordingly, the investigation began by identifying potential nucleophilic allyl sources which led to the consideration of allylBpin. The selection of this nucleophile was inspired by a 2008 report from Kobayashi and coworkers who described the allylation of ketones using allylBpin under catalysis by indium metal in water. This reaction was notable for the extraordinary catalytic efficiency of the indium which could be recovered practically unchanged at the end of the reaction. Furthermore, the authors confirmed that the indium recovered from the reaction was pure indium, not contaminated on the surface by oxides or hydroxides using a combination of SEM, EDS, and XAFS analyses. Given the aforementioned challenges with reducing sugars, it was unclear if similar reactions could be performed with native hexoses and pentoses.

Initial investigations were carried out using equimolar amount of glucose and allylBpin with 5 mol % of indium powder (325 mesh, 99.99% purity) at ambient temperature in water (1 M) to afford 1-allyl sorbitol (1) as the sole reaction product in 67% yield within 20 h (see supporting information for more details). The catalyst screening of In(0), In(I), and In(III) salts in different organic solvents (MeOH, EtOH and CH₃CN) and water mixtures revealed varying catalytic effectiveness, yielding 25%-77% of the desired product. The water soluble InCl₃, InBr₃ and In(OTf)₃ at 5 mol % loading resulted in lower yields (25%-36%) compared to partially soluble In(OAc)₃ (63% yield). Both InCl and InI performed better, yielding 55%-65% 1-allyl sorbitol. Remarkably, In(0), In₂O₃, and In(OH)₃ emerged as the most effective catalysts, achieving 50%-77% yields despite their insolubility and unclear mechanism of catalytic activity.

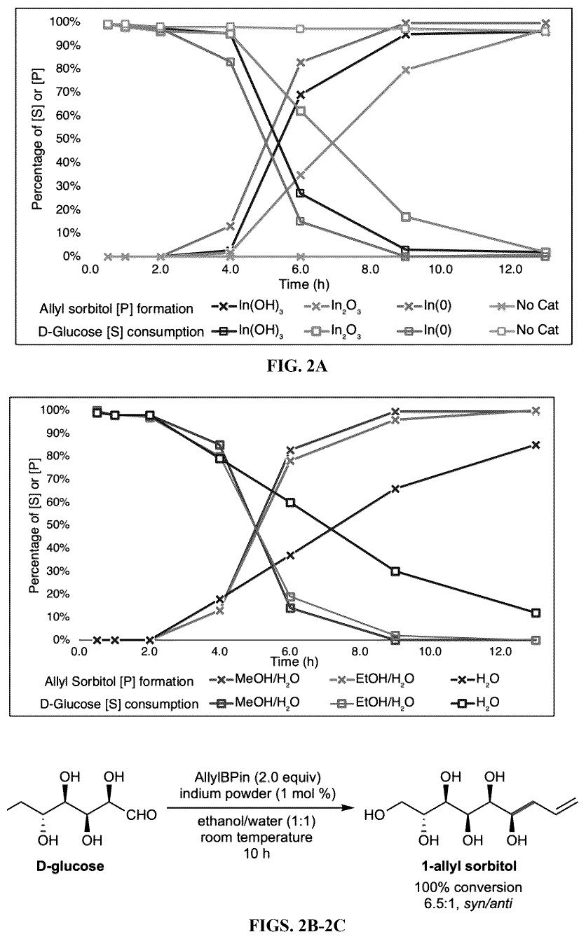

On the basis of these findings, a design of experiments (DOE) campaign was initiated focusing on In(0), In₂O₃, and In(OH)₃ as catalysts at room temperature (See supporting information for more details). Variables included catalyst loading (1 mol % to 10 mol %), allylBpin stoichiometry (1.0 equiv to 2.5 equiv), solvent concentration (2.0 M to 0.2 M), and the proportions of water/ethanol and water/acetonitrile blends (50% to 100% water). The results of the DOE revealed that the more relevant reaction factors were solvent, allylBpin stoichiometry, and concentration, while catalyst loading was less relevant and had little bearing on reaction outcomes (FIG. 2). Ultimately, In(0) (1 mol %) gave the best results giving 100% conversion in a 1:1 water/ethanol blend with 2.0 equiv of allylBpin at 2.0 M concentration. Additionally, In(0) frequently formed a single piece of indium foil which could be recovered from the reaction as observed by Kobayashi. The optimization and utilization of indium recovery is discussed in more detail in further sections.

Having successfully optimized a catalytic allylation of glucose, the stability and speciation of allylBPin in aqueous solutions was first examined. Unsurprisingly, allylboronic acid was observed by ¹H NMR analysis in D₂O even though the pinacol ester was completely insoluble. Tracking the concentration of the two species over time revealed that the hydrolysis is readily reversible with the acid and the pinacol ester quickly reaching equilibrium. Hypothetically, the boronic acid was likely the more active if not the sole active allylboron species in the reaction. This hypothesis is supported by Kobayashi's observation that allylations under anhydrous conditions were unsuccessful. To further probe this hypothesis, as well as develop more homogeneous reaction conditions, methods to generate allylboronic acid stoichiometrically without the complications of the acid-ester biphasic equilibrium were strategized. This led to an investigation of the use potassium trifluoroborate (BF₃K) salts as boronic acid precursors. Potassium trifluoroborate salts are easy to handle, bench-stable solids and allylBF₃K is commercially available and inexpensive. However, BF₃K salts do not fully hydrolyze under ambient aqueous conditions and gave poor conversion when added to the reaction mixture directly. Molander has reported that BF₃K salts can be fully hydrolyzed in water with silica gel as a fluorophile. This method worked well for the hydrolysis of allylBF₃K to obtain an aqueous solution of allylboronic acid.

With aqueous solutions of allylboronic acid readily available, allylation conditions could be evaluated. Indeed, allylboronic acid was competent under the reaction conditions achieving complete conversion with short reaction times. Furthermore, all reaction components besides indium were fully soluble in a single liquid phase, and the reaction byproducts consisted solely of boric acid thereby simplifying purification. The shorter reaction times and more homogeneous reaction conditions allowed for reliable recovery of intact indium foil.

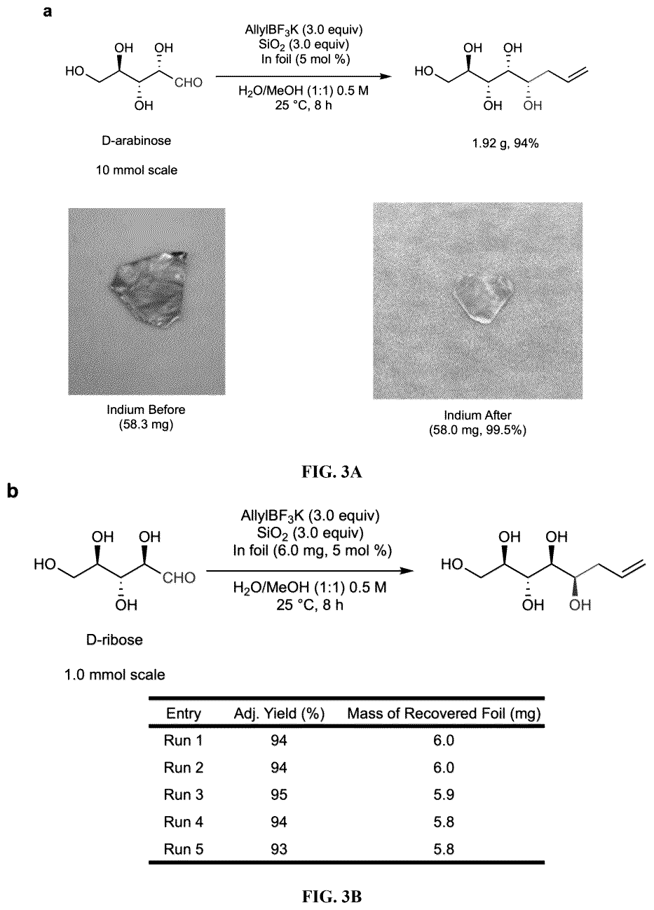

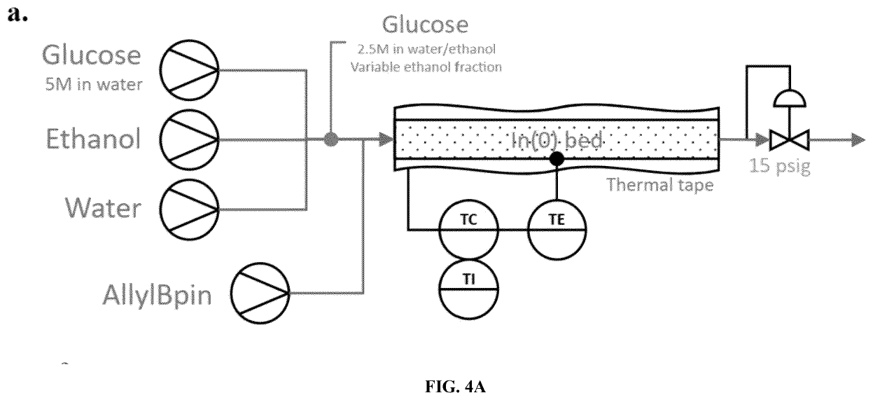

To probe the recovery and reusability of indium foil under these reaction conditions, two experiments were carried. The first was a larger scale allylation utilizing a single piece of indium foil. Arabinose was allylated on 10 mmol scale furnishing product 9 in 94% yield (FIG. 3A). The indium foil was weighed before the reaction (58.3 mg) and after the reaction (58.0 mg). The loss of only 0.3 mg over the course of the reaction represents a 99.5% recovery of the catalyst by simply removing it from the reaction mixture with forceps. The second experiment aimed to probe not only the recoverability but also the reusability of the indium foil. The allylation of ribose was carried out 5 times utilizing the same piece of indium foil, which was removed from the reaction, rinsed with water, and used in the next reaction. There was no significant change in yield between the first reaction (94%) and the fifth reaction (93%) (FIG. 3B). Additionally, the foil only lost 0.2 mg (3.33%) of its original mass over the course of the five experiments. These experiments conclusively demonstrate that indium foil is an extremely efficient catalyst for this transformation and is not only highly recoverable but also reusable.

### Example 2. Reaction Scope

With the goal of expanding this method beyond the allylation of glucose, a range of easily accessible monosaccharides were evaluated under optimized conditions. All substrate classes performed well under conditions using allylBpin with indium powder (A), allylBF₃K with indium powder (B), or allylBF₃K with indium foil (B) affording excellent yields (78-97%) of the desired allylation products. Allylsorbitol (1) from allylation of glucose was obtained in 91% yield and 6:1 d.r. after 10 h using conditions A (entry 1). In contrast to the other aldose substrates, glucose suffered from incomplete conversion and longer reaction times using allylboronic acid condition B (entry 2). Of the two conditions B and C, conditions B performed better and 1 was obtained in 80% yield with 16% remaining glucose after 18 h. Condition C did not give satisfactory conversion for glucose and as such it is not reported. Reactions of galactose (entries 3-5) and mannose (entries 6-8) were high yielding (86-93%) with galactose exhibiting notably higher diastereoselectivity (up to 10:1) than all other substrates. Although 2-deoxy-galactose was allylated with 92-94% yield, (entries 9,10) the product exhibited significantly lower diastereoselectivity (2:1 and 1.3:1) compared to other substrates. This result is in agreement with previously observed chelate-controlled diastereoselectivity in sugar substrates. Interestingly, Whitesides' indium-mediated Barbier allylation of 2-amino sugars fails under the standard conditions. A subsequently reported method from Whitesides et al. achieved the allylation of amino sugars however, only with substituted allylating agents along with aqueous acid, 4 equiv of indium, and 6 equiv of allylic bromides to achieve synthetically useful yields. In contrast, allylation conditions A resulted in 86% yield and 1.8:1 d.r. of 5 using only 2 equiv of allylBpin (entry 11). Conditions B and C, however, required 6 equiv of allylBF₃K to achieve satisfactory yields (entries 12, 13). While N-acetyl galactosamine (entry 14) performed well (84% yield and 3:1 d.r.), 4 equiv of allylBpin was used for N-acetyl glucosamine (entry 15, 87% yield and 1.4:1 d.r.). Aldopentoses (ribose, arabinose, and xylose) exhibited high reactivity with excellent isolated yields (entries 16-25, 82-97%) and generally lower reaction times. Fructose, a ketohexose, was also allylated with excellent yields (entries 26-28, 92-97% yield). Polyfunctional substrates such as glucuronolactone (entry 29) and glucuronic acid (entry 30) were allylated to furnish products 13 (92% yield, 4:1 d.r.) and 14 (84% yield, 3.5:1 d.r.) with complete selectivity for allylation at the aldehyde versus the acid or ester. The broad substrate scope highlights the power and versatility of this catalytic route for carbohydrate allylation beyond the synthesis of allylsorbitol for Millad® NX® 8000.

### Example 3. Bayesian Optimization in Flow

Finally, to make this new process suitable for industrial applications, the remarkable recoverability and reusability of indium by incorporating In(0) as a stationary catalyst in a packed-bed flow reactor were leveraged and thereby achieved the allylation of native glucose in a scalable flow-based format (FIG. 4A). An optimization campaign was executed considering the 4-dimensional design space of operating conditions: total flow rate (glucose solution and allylBPin stream), equiv of allylBPin, temperature, and ethanol fraction in the glucose solution. Given that: (1) side reactions are present (i.e., the formation of propene), (2) complete information on the mechanism is not available, and (3) four phases are present in the reactor (two liquid phases, a solid catalytic phase, and a gas phase of byproducts), a deterministic phenomenological model could not be built. Thus, Bayesian optimization (BO) was selected as a powerful, and efficient tool to obtain the best operating conditions for maximum yield from a set of designed experiments (details of the BO are available in the SI).

At the end of the optimization campaign, the best operating conditions were experimentally obtained for maximum yield [100% total yield, 8:1 d.r., i.e., 88.9% syn isomer yield (very similar d.r. to the current industrial process)]: low total flowrate (17.5 μL/min), high temperature (80° C.), high equiv of allylBPin (2.5), and an intermediate value for the ethanol fraction (40%). The yield was maximized by lowering the total flow rate as much as possible, independent of the values of the other three parameters (FIG. 4B). Similarly, high temperatures and large amounts of allylBPin were preferred, as expected, independent of the other parameters (FIGS. 4c and 4d, respectively). There was no clear trend for the ethanol fraction as an independent variable (FIG. 4E), indicating a coupled or minor effect compared to the other variables. These results suggest that potential side reactions are relatively slow in the explored design space. The yield-optimal conditions were tested on an extended run over 5 h, from which 1.03 g of product were obtained, corresponding to an isolated yield of 86.4% syn isomer, showing the robustness of the optimal conditions over time.

Productivity (space-time yield) is not maximized under the conditions of optimum reaction yield as the productivity optimum (0.735 g/h of the syn isomer) is located at a higher total flow rate (52.5 μL/min), a higher temperature (80° C.), fewer equiv of allylBPin (1.0), and a higher value for the ethanol fraction (50%). Productivity and yield are in fact competing objectives; the corresponding Pareto front was found without the need for additional experiments (FIG. 4F). As shown in FIG. 4G, FIG. 4H, and FIG. 4I, small deviations from the Pareto-optimal conditions will not markedly affect productivity and yield, making the inherently sustainable catalytic process highly robust and thus favorable for scaled industrial application.

### Example 4. Method of Bayesian Optimization in Flow

The lab-scale tubular reactor used for flow experiments (FIG. 4A) was built in the lab using stainless-steel tubing (0.175″ ID, 78.5 mm length) and standard threaded connections for gas systems. Fluids (glucose 5M solution in water, allylBpin, water, and ethanol) were fed into the reactor using PHD ULTRA™ syringe pumps from Harvard Apparatus and PTFE tubing ( 1/32″ ID). Indium (100 mesh) was mixed with glass beads (100 μm dia.) to constitute the catalytic bed that was packed in the tube. Catalyst loading was selected via preliminary experiments (see SI) aiming to reduce the amount of indium used. During all experiments, the reactor was kept at set temperatures via thermal tape (on external wall of the tube) and a PID feedback controller with a K-type thermocouple. Pressure was set at the outlet of the steel tube using a Back-Pressure Regulator set at 15 psig.

The optimal lab-scale-process conditions should provide good yield (objective to be maximized)—corresponding to low separation costs at industrial scale. The experimental optimization campaign is driven by BO (see Supplementary Information for mathematical details) in a selected, finite design space. The design space is constructed as a four-dimensional domain: (i) total flowrate, \dot{Q}: 17.5-52.5 μL/min; (ii) temperature, T: 50-80° C.; (iii) equiv of allylBpin (w.r.t. glucose), b: 1.0-2.5; (iv) ethanol fraction in the inlet glucose solution, x_{EtOH}: 30-50%. In practice, an initialization set of experimental conditions (usually randomized, e.g., via Latin Hypercube Sampling) is run and the GP underlying the methodology is trained on this dataset. By maximizing an acquisition function, it is possible to obtain a new set of experimental conditions to test. The GP is re-trained on the set of data augmented with the new point and a novel experiment is suggested again by the algorithm. The GP is re-trained each new experiment to continuously acquire information for a fast optimization campaign.

Productivity—related to industrial process revenue—was observed after completing the yield optimization campaign, extending the optimization to two competing objectives. This is possible because yield and productivity are strongly related mathematically, despite being competing objectives in general. Productivity, [symbol] , and yield, Y, are rarely possible to be maximize under the same conditions, posing a multi-objective optimization problem. The aim is to obtain a Pareto front displaying the trade-off between the two objectives at their optimal values. Nevertheless, [symbol] =m·Y and the proportionality constant, m, is a known function of the design space variables, X:

$$𝒫 ( X ) = {\underbrace{M {W}_{prod} {c}_{g l u}^{s t o c k} {\dot{Q}}_{g l u} ( X )}}_{m ( X )} \cdot Y ( X ) \qquad ( 1 )$$

Where MW_{prod} is the molecular weight of the product (222 g/mol),

$${c}_{g l u}^{s t o c k}$$

the concentration of glucose in the stock solution (5 M), and \dot{Q}_{glu} is the volumetric flowrate of the glucose stock solution, which is again a function of the optimization parameters:

$${\dot{Q}}_{glu} ( X ) = \frac{Q}{{c}_{glu}^{stock} \cdot ( \frac{b}{{c}_{B p i n}^{stock}} + \frac{1}{{c}_{glu}^{mix}} )} \qquad ( 2 )$$

where

$${c}_{B p i n}^{s t o c k}$$

is the concentration of pure allylBpin (5.33 M) and

$${c}_{g l u}^{m i x}$$

is the concentration of glucose in the water-ethanol solution (2.5 M) to be mixed with allylBpin.

Y and [symbol] should be both maximized to obtain Pareto-optimal conditions. This means that points on the Pareto front are characterized by a productivity that is the maximum possible for each value of yield (and vice versa, by the maximum value of yield for each value of productivity). The multi-objective optimization problem can thus be recast in a simpler form as:

$$\{ \begin{matrix}{arg max}_{X} 𝒫 ( X ) \\ Y ( X ) given , ∀ Y\end{matrix} \qquad ( 3 )$$

This is, by substitution argmax m(X)·Y(X), and (as Y(X) is given) the Pareto-optimal condition can be found by solving the constrained maximization argmax m(X). As m(X) is a known function (Eq. 1), Pareto-optimal conditions are obtained by finding the point of maximum glucose flowrate on an iso-surface of Y in the design space. Exploiting this relationship, the full, global Pareto front can be estimated solely from the yield GP (which provides an easy-to-evaluate function Y(X)), without additional experiments.

### Example 5. Reaction Setup

All reactions were performed in non-dried glassware under ambient atmosphere unless otherwise indicated. Room temperature (rt) was approximately 23-28° C. Glass vials, flasks, and test tubes were purchased from Chemglass. Reaction optimization was carried out using the GreenHouse Plus Parallel Synthesizer™ and Heidolph Hei-PLATE Hotplate Stirrer. Tisch Scientific syringe filter was used for filtration.

### Example 6. NMR Spectroscopy

¹H, ¹³C, ¹⁹F, and ¹¹B NMR spectra were recorded in D₂O (purchased from Cambridge Isotope) on a Bruker Acsend™ 600 MHz (600 MHz, ¹H; 151 MHz, ¹³C) spectrometer. Spectra are referenced to HDO (δ=4.79 ppm, 1H) and ¹³C spectra are unreferenced. Chemical shifts are reported in parts per million (ppm) and multiplicities are indicated by s (singlet), d (doublet), t (triplet), q (quartet), quint (quintet), m (multiplet), and br (broad). Coupling constants (J) are reported in hertz (Hz), integration is provided, and assignments are indicated. Assignments were obtained by reference to COSY, HSQC, and HMBC correlations. ¹H Q-NMR spectroscopic analysis was performed using 900 pulse, d1=60 s, and referenced to the indicated internal standard. ¹³C spectra were acquired using a standard pulse (p1=10 μs) and relaxation delay (d1=2 s).

### Example 7. Mass Spectrometry

Mass spectrometry was performed by the University of Illinois Mass Spectrometry Laboratory. Mass spectrometric data was collected on a Waters Q-TOF Ultima (ESI) spectrometer, a Waters Synapt G2-Si spectrometer (ESI), a Waters Quattro Ultima spectrometer (ESI), or a Waters 70-VSE spectrometer (EI). Low-resolution spectral data are reported as (mass, intensity), and high-resolution data are reported as calculated and measured masses to 10⁻⁴ mass accuracy.

### Example 8. Infrared Spectroscopy

Infrared spectra were recorded on a Perkin-Elmer UATR-2 FT-IR spectrophotometer. Peaks are reported in cm⁻¹ with relative intensities indicated: s (strong, 67-100%); m (medium, 34-66%), w (weak, 0-33%).

### Example 9. Melting Points

Melting points (m.p.) were determined on an SRS DigiMelt MPA160 capillary melting point apparatus in sealed tubes and are corrected.

### Example 10. Chromatography

Silica plug filtrations were performed using Silicycle SiliaFlash® P60 (40-63 μm particle size, 230-400 mesh) (SiO₂). Unless otherwise specified, “silica” refers to P60 grade silica gel. Analytical thin-layer chromatography was performed on Merck silica gel 60 F254 plates. TLC plates used acetone-water-butanol mixture as eluent and were visualized by treatment with anisaldehyde stain. Analytical high-performance liquid chromatography (HPLC) was performed using a Supelcosil™ LC-18 HPLC column (4.6×250 mm, 5.0 μm particle size) and Shimadzu (low temperature) evaporative light scattering detector (ELSD). 0.20 μm Tisch Scientific HPLC grade syringe filters were used for sample filtration.

### Example 11. Solvents

All reaction solvents were used as received from the following sources: tetrahydrofuran (Fischer, HPLC grade), hexanes (Fischer, HPLC grade), diethyl ether (Fischer, BHT stabilized ACS grade), methylene chloride (Fischer, unstabilized HPLC grade), DMF (Fischer, HPLC grade), acetonitrile (ACS grade), Millipore water or water (Aqua Solutions, Inc., Distilled ASTM type II), methanol (Macron, ACS grade). When necessary, solvents were dried by percolation through two columns packed with neutral alumina under positive pressure of argon. Solvents for filtration, transfers, and chromatography were purchased from commercial sources and used as received.

### Example 12. Chemicals

Indium powder (325 mesh, 99.99%) was sourced from Oakwood Chemical. Indium powder (100 mesh, 99.99%) was sourced from Strem Chemical. Indium foil (0.127 mm thickness, 99.99%) was sourced from Sigma-Aldrich and was added to the reaction as a single piece weighing the indicated mass. Other indium sources: InCl₃ (99.99%, Strem Chemical), InBr₃ (Sigma Aldrich), In(OAc)₃ (99.99%, Strem chemical), In(OTf)₃ (Sigma Aldrich), InI (99.99%, AmBeed), InCl (97%, AmBeed), In₂O₃ (Sigma Aldrich), and In(OH)₃ (99.99% metal basis, ThermoScientific). The hygroscopic InCl₃, InBr₃, and In(OTf)₃, were stored in glove box, and InCl was stored in the freezer of the glove box. Allylboronic acid pinacol ester (97%, stabilized with phenothiazine) was sourced from AmBeed and stored in a freezer (−20° C.). Potassium allyltrifluoroborate was sourced from Aaron Chemicals and was found to be 89% pure by Q-NMR. Note: purity was found to vary significantly by supplier. Caffeine was used as an internal standard and purchased from Sigma Aldrich (99%). The following commercial reagents were used as received: D-glucose, D-galactose, D-mannose, N—Ac-D-mannosamine, N—Ac-D-glucosamine, N—Ac-D-galactosamine, D-arabinose, D-ribose, D-xylose, D-fructose, 2-deoxy-D-galactose, D-glucuronolactone, and D-glucuronic acid.

### Example 13. Allylation of Carbohydrates with Allylboronic Acid Pinacol Ester (AllylBpin)

### Reaction Optimization

The synthesis of 1-allyl sorbitol followed Whitesides's Barbier-type allylation, as represented by the following scheme:

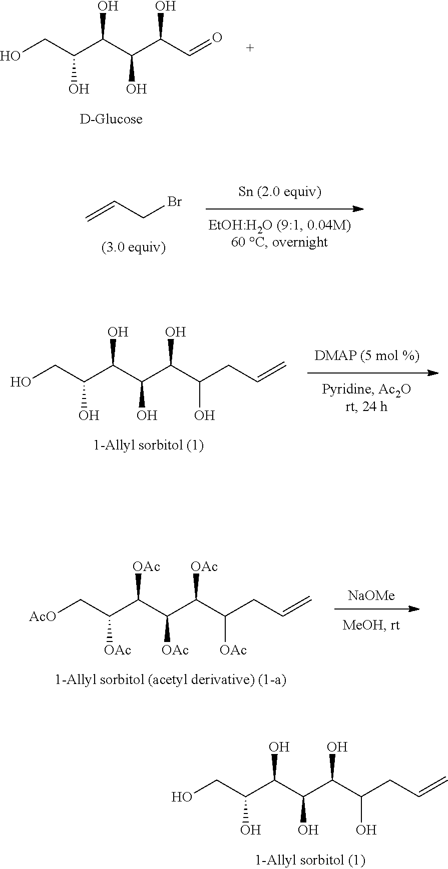

1-Allyl sorbitol (1) was synthesized following Whitesides's method (Scheme 1). To a two-necked round-bottom flask was added 5 mmol of D-glucose, 10 mmol of tin powder, and 125 mL of ethanol-water (1:1) under a nitrogen atmosphere. Then, 15 mmol of allyl bromide was added, and the suspension was refluxed until completion (20 h) as determined by TLC (butanol-acetone-water 4:5:1). The resulting solution was highly acidic (pH≈1) and was neutralized with 6 N NaOH followed by vacuum filtration through a Celite bed. The solvent was evaporated under reduced pressure yielding a colorless syrup which was used for the next step without further purification.

The product was dissolved in 25 mL of pyridine, then 9 mL of acetic anhydride and 30 mg of DMAP (4-dimethylaminopyridine) were added. The reaction mixture was stirred for 24 h at room temperature and then purified by silica gel column chromatography. The acetylated 1-allyl sorbitol was obtained as a colorless solid (1-a, 1.23 g, 52% yield). The compound spectra are consistent with the literature report.³

Deacetylation was carried out following a literature procedure.⁴ Under a nitrogen atmosphere, the acetylated 1-allyl sorbitol (948 mg, 2 mmol) was dissolved in anhydrous MeOH. NaOMe (0.1 equiv. per —OH group) was then added at 0° C., and the reaction mixture was gradually warmed up to room temperature. Upon completion, the reaction was neutralized using Amberlyst-15 and filtered. The filtrate was concentrated under reduced pressure yielding a colorless syrup which was dissolved in 10 mL of distilled water and lyophilized to obtain allyl sorbitol (1) as a white solid.

¹H NMR: (600 MHz, D₂O) δ 5.87 (ddt, J=16.9, 10.2, 7.0 Hz, 1H, HC(8)), 5.20-5.10 (m, 2H, HC(9)), 3.95 (dd, J=6.1, 2.3 Hz, 1H, HC(2)), 3.85-3.79 (m, 2H, HC(1,6)), 3.79-3.74 (m, 1H, HC(4)), 3.71-3.68 (m, 1H, HC(3)), 3.67-3.61 (m, 2H, HC(1,5)), 2.41-2.28 (m, 2H, HC(7)).

¹³C NMR: (151 MHz, D₂O) S 134.7 (C(8), 117.6 (C(9), 73.8 (C(5), 71.0 (C(3), 71.0 (C(4), 70.3 (C(6), 69.9 (C(2), 62.8 (C(1), 37.4 (C(7).

### Experimental and Analytical Procedures for Reaction Optimization in Batch

Optimizing this reaction required a systematic workflow for experimental setup of reactions in the Greenhouse. Reaction tubes were cleaned using detergent and physical abrasion to remove bulk residues of indium metal and other organics. They were then soaked in aqua regia, rinsed with deionized water, then soaked in a base bath (KOH/isopropyl alcohol), and lastly rinsed with deionized water and thoroughly dried in an oven.

The reaction tubes were loaded into the 24-position rack and charged with oven-dried stir bars (soaked in aqua regia, quenched/rinsed, and dried between each use). The tube rack was allowed to cool, then the insoluble/partially soluble solid catalysts (In metal, In₂O₃, In(OH)₃, In(OAc)₃, InCl, and InI) were added. Solvent was added to each tube followed by stock solutions of D-glucose and the soluble catalysts (In(OTf)₃, InCl₃, and InBr₃). Finally, allylBpin was added to each reaction. All liquid additions (including solvent) were made with volumetric pipets, and stock solutions of reactants were mixed by charging a desired reactant gravimetrically into a volumetric flask, diluting, then dosing an aliquot using volumetric pipets.

The tube rack was then transferred back into the reactor body, and the system was sealed with a rubber pipet bulb over the gas port. The reactor was placed onto a stir plate set to 600 rpm at room temperature. After 20 h±15 min (unless otherwise mentioned), 1 mL of a stock solution containing 0.25 mmol of caffeine internal standard with respect to 0.5 mmol glucose was added using a volumetric pipet. The reactions were then stirred continuously while an aliquot was removed and diluted into 1.0 mL of spectroscopic grade 60:40 water-methanol before filtering through a 0.2 m syringe filter. The samples were then analyzed using the described RP-HPLC method.

### Initial Survey of Catalysts and Solvents

Preliminary experiments were conducted using 0.5 mmol of D-glucose, 0.5 mmol of allylBpin, and 5 mol % of indium powder (325 mesh, 99.99% purity) at ambient temperature in water (1 M). This reaction afforded 1-allyl sorbitol (1) as the sole product in 67% yield after 20 h. The moderate yield is attributed to competitive hydrolysis of the allylic nucleophile forming propene gas.

Catalyst and solvent survey began with equimolar amounts of D-glucose and allylBpin at 1 M concentration and room temperature. The following catalysts were evaluated at 5 mol %: In metal (325 mesh), InI, InCl, InCl₃, InBr₃, In(OAc)₃, In(OTf)₃, In₂O₃, and In(OH)₃. The following solvents were surveyed: water, water/ethanol (1:1), water/methanol (1:1), and water/acetonitrile (1:1).

In metal, In₂O₃, and In(OH)₃ emerged as the most effective catalysts despite their insolubility and unclear mechanism of catalytic activity. InCl and InI (which disproportionate in water to In(III) and In (0)) exhibited moderate effectiveness while the fully soluble InCl₃, InBr₃, and In(OTf)₃ were the least effective. Control experiments confirmed negligible reactivity in the absence of catalyst and no catalytic activity from boric acid alone.

### Indium Recovery and Reusability

Under the reaction conditions, indium foil forms within 4 h when indium powder (325 or 100 mesh) was used. No significant loss of mass or catalytic activity was observed from the recovered indium foil.

The allylation of D-ribose was conducted following Representative Procedure B using a 6.0 mg piece of indium foil. The reaction was complete after 8 h, and the indium foil was removed with forceps, rinsed with distilled water, and weighed. The reaction was worked up and the product characterized in the same manner as (product 8). This process was repeated four times using the recovered indium foil as the catalyst in the following reaction.

### Hydrolysis of AllylBpin in Water

¹H NMR experiments were conducted to determine the species present in the aqueous layer of the biphasic allylBpin/water mixture. With caffeine as an internal standard, allylBpin (18 mg, 0.1 mmol) was charged into an NMR tube containing D₂O (0.5 mL). The tube was shaken vigorously for 30 seconds before immediate injection into the NMR, and one scan was collected every minute over the course of an hour. The data shows the concentration of the allylBpin drops rapidly over the first few min. This is primarily due to the insolubility of the allylBpin which can be seen forming a separate phase above the aqueous phase and out of the signal window of the RF coils.

Additionally, hydrolysis of the pinacol ester to allylboronic acid occurs rapidly with a significant amount already present during the first scan. The concentration of allylboronic acid continues to increase slightly until reaching a plateau when the allylBpin has fully phase-separated. Further shaking of the sample results in no further increase in this concentration. This maximum concentration of allylboronic acid (ca. 0.1 M) is independent of the total amount of allylBpin above this concentration. The addition of pinacol to this solution causes a decrease in the concentration of allylboronic acid. This behavior indicates that there is a solubility equilibrium with dissociation between the allylBpin and its hydrolysis products, allylboronic acid and pinacol.

### Example 14. Representative Allylation Procedure A (AllylBpin, In Powder): (2R,3R,4R,5S)-Non-8-ene-1,2,3,4,5,6-hexaol (1) from D-Glucose

The following scheme was followed for this representative example:

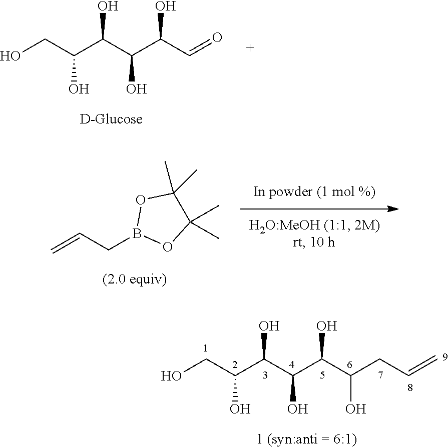

A 1-dram vial equipped with a magnetic stir bar (2×1 mm) was charged with indium powder (325 mesh, 1 mg, 1 mol %) and D-glucose (180 mg, 1.0 mmol) followed by addition of 1:1 mixture of methanol/water (0.50 mL). Subsequent addition of allylBpin (375 μL, 2.0 mmol, 2.0 equiv) to the reaction mixture resulted in the formation of a biphasic layer. The reaction mixture was stirred for 10 h at 23° C. resulting in a clear, monophasic solution. The solution was filtered through a fritted funnel with successive rinsing of the vial with water (20 mL) and transferred to a 100-mL, round-bottom flask. The filtrate was concentrated under reduced pressure (55-60° C., 15 mmHg) to afford a clear, colorless syrup. The syrup was dissolved in MeOH (40 mL) and evaporated under reduced pressure (55-60° C., 15 mmHg), and the process was repeated six times (to remove boric acid as trimethyl borate) to obtain a semi-solid residue. The residue was then dissolved in 5 mL of water and was lyophilized (0.2 mmHg) for 2 d to afford a white solid. The solid was then sonicated with CH₂Cl₂ (20 mL) for 5 min and then filtered through a fritted funnel (to remove pinacol) to yield a residue of 201 mg (97% purity (Q-NMR), 88% yield w.r.t. purity) of (1) as a white solid.

Data for (1):

d.r.: 6:1 (C(5)/C(6); syn anti)

m.p.: 96-99° C.

¹H NMR: (600 MHz, D₂O) δ 5.87 (ddt, J=16.9, 10.2, 7.0 Hz, 1H, HC(8)), 5.20-5.10 (m, 2H, HC(9)), 3.95 (ddd, J=6.1, 2.3, 0.7 Hz, 1H, HC(2)), 3.85-3.79 (m, 2H, HC(1,6)), 3.79-3.74 (m, 1H, HC(4)), 3.71-3.68 (m, 1H, HC(3)), 3.67-3.61 (m, 2H, HC(1,5)), 2.41-2.28 (m, 2H, HC(7)).

¹³C NMR: (151 MHz, D₂O) δ 134.7 (C(8), 117.6 (C(9), 73.8 (C(5), 71.0 (C(3), 71.0 (C(4), 70.3 (C(6), 69.9 (C(2), 62.8 (C(1), 37.4 (C(7)).

Q-NMR: 97% (caffeine, D₂O)

IR (neat): 3318 (m), 2931 (w), 1738 (s), 1366 (m), 1217 (m), 1076 (s), 1020 (s), 912 (w), 627 (w), 542 (w).

HRMS: Calcd. for C₉H₁₇O₆(M-H)⁻: 221.1025; found 221.1028.

### Example 15. Representative Allylation Procedure A (AllylBpin, In Powder): (3S,4R,5R)-2-Allylhexane-1,2,3,4,5,6-hexaol (12) from D-Fructose

The following scheme was followed for this representative example:

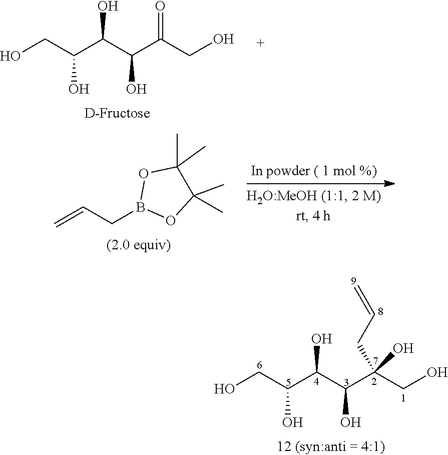

Following Representative Procedure A, a solution of D-fructose (180 mg, 1.0 mmol) with all other reaction components was stirred for 4 h whereupon the identical workup afforded 211 mg (97% purity (Q-NMR), 92% yield w.r.t. purity) of (12) as a white solid.

Data for (12):

d.r.: 4:1 (C(2)/C(3); syn anti)

m.p.: 97-102° C.

¹H NMR: (600 MHz, D₂O) δ 5.87 (ddt, J=17.4, 10.0, 7.3 Hz, 1H, HC(8)), 5.22-5.14 (m, 2H, HC(9)), 3.87 (s, 1H, HC(3)), 3.85-3.77 (m, 2H, HC(4, 6)), 3.72 (ddd, J=8.8, 6.3, 2.8 Hz, 1H, HC(5)), 3.67-3.58 (m, 3H, HC(1,6)), 2.49-2.32 (m, 2H, HC(7)).

¹³C NMR: (151 MHz, D₂O) δ 132.6 (C(8)), 119.0 (C(9)), 76.6 (C(2)), 71.2 (C(5)), 70.8 (C(3)), 69.6 (C(4)), 63.5 (C(1)), 62.9 (C(6)), 37.8 (C(7)).

Q-NMR: 97% (caffeine, D₂O)

IR (neat): 3291 (m), 2937 (w), 2890 (w), 1737 (w), 1640 (w), 1409 (m), 1288 (s), 1229 (w), 1065 (s), 1031 (s), 915 (m), 878 (m), 680 (w), 570 (w).

HRMS: Calcd. for C₉H₁₇O₆(M-H)⁻: 221.1025; found 221.1028.

### Example 16. Allylation of Carbohydrates with Potassium Allyltrifluoroborate (AllylBF₃K)

### Reaction Optimization

In surveying conditions for the allylation of glucose utilizing allylBF₃K, two main factors were considered: solvent blends and indium particle size. It was found that achieving full conversion was quite challenging as the hydrolysis of the presumed active allylindium nucleophile was competitive with productive allylation. For this reason, optimization efforts were focused on maximizing conversion over all other factors. Optimization began with a survey of solvent blends.

Pure water performed with worst of all the solvents surveyed with an assay conversion of only 76%. Equal volume blends of both water/ethanol and water/methanol were next evaluated and surprisingly there was a noteworthy difference between water-ethanol (80% conv.) and water-methanol (99% conv.). Since a clear advantage was observed with water-methanol, it was next evaluated how changing the solvent ratios would impact the conversion. It was found that either increasing or decreasing the volume of methanol negatively impacted the reaction conversion. The reaction concentration could not be increased with this fixed solvent ratio since the minimum volume of water is determined by the allylBF₃K hydrolysis conditions detailed in the next section.

Having established that methanol/water (50:50) was the ideal solvent for this transformation, the effect of indium particle size on the reaction was then examined.

Catalyst loading was evaluated with a consistent particle size (325 mesh) and it was found that increasing the loading from 2.5 mol % to 5 mol % showed marginal benefits, but further increasing the loading to 10 mol % showed no additional increase in reaction conversion. With the catalyst loading fixed at 5 mol %, three indium particle sizes were tested: 100 mesh, 325 mesh, and a single piece of indium foil. While the two indium powders exhibited no significant differences, the indium foil garnered a profound increase in the reaction conversion at the expense of longer reaction times. When substrates with shorter reaction times were tested under the optimized conditions, both indium powder and foil were achieved satisfactory reaction conversions, presumably due to the increased concentration of open-chain sugar in solution. For this reason, most substrate reactions are reported with both indium powder and foil.

### General AllylBF₃K Hydrolysis Procedure

The following synthetic scheme was followed:

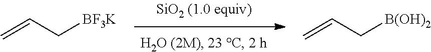

A 1-dram vial equipped with a stir bar (2×1 mm) was charged with allylBF₃K (444 mg, 3.00 mmol, 3 equiv) followed by silica gel (SiO₂, 180 mg, 230-400 mesh, 3.00 mmol, 3.0 equiv) and then distilled H₂O (1.5 mL, 2.0 M). The suspension was vigorously stirred for 2 h whereupon the solution turned pale yellow, and the allylBF₃K dissolved. After the elapsed time, the solution was filtered using a syringe filter (0.45 μm) to obtain approximately 1 mL of clear, pale-yellow filtrate (ca. 1.5 mmol, 1.5 equiv of allylboronic acid).

Note: The filtration can be made easier by inverting the syringe for 5 min prior to filtering to allow the silica to settle.

### Example 17. Representative Allylation Procedure B (AllylBF₃K, In Foil): (2R,3R,4R,5S,6R)-Non-8-ene-1,2,3,4,5,6-hexaol (1) from D-Glucose

The following synthetic scheme was followed:

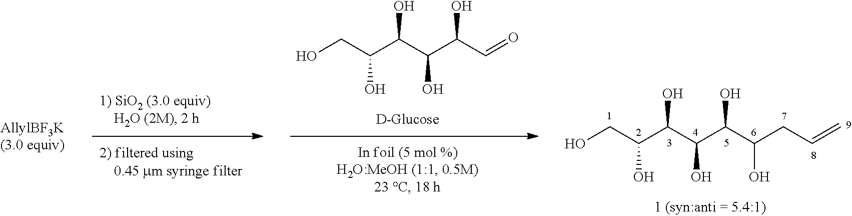

A 1-dram vial equipped with a stir bar (2×1 mm) was charged with allylBF₃K (444 mg, 3.00 mmol, 3.0 equiv) followed by silica gel (SiO₂, 180 mg, 230-400 mesh, 3.00 mmol, 3.0 equiv) and then distilled H₂O (1.5 mL, 2.0 M). The suspension was vigorously stirred for 2 h whereupon the solution turned pale-yellow and the allylBF₃K dissolved. After the elapsed time, the solution was filtered using a syringe filter (0.45 μm) to obtain approximately 1 mL of clear, pale-yellow filtrate (ca. 1.5 mmol, 1.5 equiv of allylboronic acid). A new 1-dram vial equipped with a stir bar (2×1 mm) was charged with indium foil (5.4 mg, 5 mol %) and D-glucose (180 mg, 1.00 mmol) followed by simultaneous addition of the filtrate (1 mL, 1.0 M) and MeOH (1 mL, 1.0 M), and the suspension was stirred for 18 h at 23° C. The suspension was transferred to a 100 mL round-bottomed flask with successive rinsing of the vial with MeOH (20 mL). The solution was concentrated under reduced pressure (60° C., 15 mmHg) to afford a clear, colorless syrup. This was dissolved in MeOH (5 mL) and filtered through a pad of silica (5 mm×3 mm diameter) and the filter cake was washed with MeOH (50 mL). The clear, colorless filtrate was concentrated under reduced pressure (60° C., 15 mmHg) to obtain a clear, colorless syrup. This syrup was dissolved in a minimal amount of MeOH (~5 mL), filtered through a syringe filter (0.22 μm), and the filtrate was concentrated under reduced pressure (60° C., 15 mmHg) to obtain a clear, colorless syrup. This syrup was dissolved in distilled H₂O (~10 mL) and filtered through a syringe filter (0.45 μm). The filtrate was lyophilized (0.2 mmHg) for 1-2 d to yield 218 mg (82% purity (Q-NMR), 80% yield w.r.t purity) of (1) as a hygroscopic white solid. 15% unreacted D-glucose was identified by ¹H NMR.

Data for (1):

d.r.: 5.4:1 (C(5)/C(6); syn anti)

¹H NMR: (600 MHz, D₂O) δ 5.87 (ddt, J=16.9, 10.2, 7.0 Hz, 1H, HC(8)), 5.20-5.10 (m, 2H, HC(9)), 3.95 (ddd, J=6.1, 2.3, 0.7 Hz, 1H, HC(2)), 3.85-3.79 (m, 2H, HC(1,6)), 3.79-3.74 (m, 1H, HC(4)), 3.71-3.68 (m, 1H, HC(3)), 3.67-3.61 (m, 2H, HC(1,5)), 2.41-2.28 (m, 2H, HC(7)).

¹³C NMR: (151 MHz, D₂O) δ 134.7 (C(8), 117.6 (C(9), 73.8 (C(5), 71.0 (C(3), 71.0 (C(4), 70.3 (C(6), 69.9 (C(2), 62.8 (C(1), 37.4 (C(7).

Q-NMR: 82% (caffeine, D₂O)

HRMS: Calcd. for C₉H₁₇O₆ (M-H)⁻: 221.1025. Found: 221.1022.

### Example 18. Representative Allylation Procedure B (AllylBF₃K, In Foil): (2S,3S,4R,5R)-2-Allylhexane-1,2,3,4,5,6-hexaol (12) from D-Fructose

The following synthetic scheme was followed:

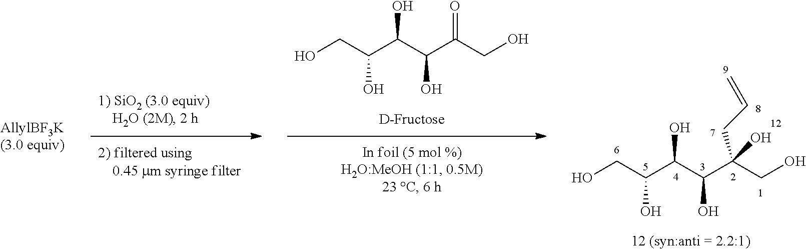

Following Representative Procedure B, a solution of D-fructose (180 mg, 1.00 mmol) with all other reaction components was stirred for 6 h whereupon the identical workup afforded 236 mg (91% purity (Q-NMR), 97% yield w.r.t purity) of (12) as a colorless glass.

Data for (12):

d.r.: 2.2:1 (C(2)/C(3); syn anti)

¹H NMR: (600 MHz, D₂O) δ 5.87 (ddt, J=17.4, 10.0, 7.3 Hz, 1H, HC(8)), 5.22-5.14 (m, 2H, HC(9)), 3.87 (s, 1H, HC(3)), 3.85-3.77 (m, 2H, HC(4, 6)), 3.72 (ddd, J=8.8, 6.3, 2.8 Hz, 1H, HC(5)), 3.67-3.58 (m, 3H, HC(1,6)), 2.49-2.32 (m, 2H, HC(7)).

¹³C NMR: (151 MHz, D₂O) δ 132.6 (C(8)), 119.0 (C(9)), 76.6 (C(2)), 71.2 (C(5)), 70.8 (C(3)), 69.6 (C(4)), 63.5 (C(1)), 62.9 (C(6)), 37.8 (C(7)).

Q-NMR: 91% (caffeine, D₂O)

HRMS: Calcd. for C₉H₁₇O₆ (M+Na)⁺: 245.1001. Found: 245.0994.

### Example 19. Representative Allylation Procedure C (AllylBF₃K, In Powder): (2R,3S,4R,5S,6R)-Non-8-ene-1,2,3,4,5,6-hexaol (2) from D-Galactose

The following synthetic scheme was followed:

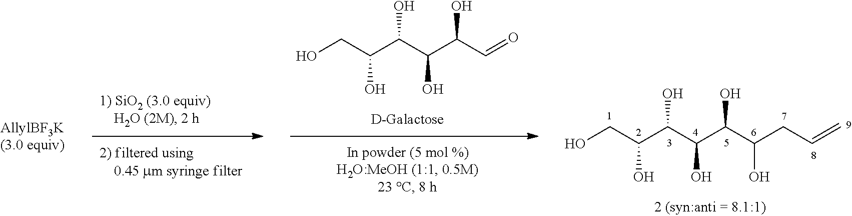

A 1-dram vial equipped with a stir bar (2×1 mm) was charged with allylBF₃K (444 mg, 3.00 mmol, 3.00 equiv) followed by silica gel (SiO₂, 180 mg, 230-400 mesh, 3.00 mmol, 3.00 equiv) and then distilled H₂O (1.5 mL, 2.0 M). The suspension was vigorously stirred for 2 h whereupon the solution turned pale-yellow and the allylBF₃K dissolved. After the elapsed time, the solution was filtered using a syringe filter (0.45 μm) to obtain approximately 1 mL of clear pale-yellow filtrate (ca. 1.5 mmol, 1.5 equiv of allylboronic acid). A new 1-dram vial equipped with a stir bar (2×1 mm) was charged with indium powder (100 mesh, 5.4 mg, 5 mol %) and D-galactose (180 mg, 1.00 mmol) followed by simultaneous addition of the filtrate (1 mL, 1.0 M) and MeOH (1 mL, 1.0 M) and the suspension was stirred for 8 h at 23° C. The suspension was transferred to a 100 mL round-bottomed flask with successive rinsing of the vial with MeOH (20 mL). The solution was concentrated under reduced pressure (60° C., 15 mmHg) to afford a clear, colorless syrup. The syrup was dissolved in MeOH (5 mL) and filtered through a pad of silica (5 mm×3 mm diameter) and the filter cake was washed with MeOH (50 mL). The clear, colorless filtrate was concentrated under reduced pressure (60° C., 15 mmHg) to obtain a clear, colorless syrup. The syrup was dissolved in a minimal amount of MeOH (~5 mL), filtered through a syringe filter (0.22 μm), and the filtrate was concentrated under reduced pressure (60° C., 15 mmHg) to obtain a clear, colorless syrup. The syrup was dissolved in distilled H₂O (~10 mL) and filtered through a syringe filter (0.45 μm). The filtrate was lyophilized (0.2 mmHg) for 1-2 d to yield 233 mg (85% purity (Q-NMR), 89% yield w.r.t purity) of (2) as a white solid. 4% unreacted D-galactose was identified by ¹H NMR.

Data for (2):

d.r.: 8.1:1 (C(5)/C(6); syn anti)

¹H NMR: (600 MHz, D₂O) δ 5.93-5.83 (ddt, J=17.5, 10.1, 7.0 Hz, 1H, HC(8)), 5.18 (dd, J=17.4, 1.2 Hz, 1H, HC(9)), 5.14 (dd, J=10.2, 1.0 Hz, 1H, HC(9)), 3.95 (t, J=5.8 Hz, 1H, HC(2)), 3.85 (ddd, J=8.0, 6.7, 4.3 Hz, 1H, HC(6)), 3.78 (d, J=9.3 Hz, 1H, HC(3)), 3.74 (d, J=6.7 Hz, 1H, HC(5)), 3.71-3.64 (m, 3H, HC(1,4)), 2.42 (ddd, J=14.5, 7.0, 4.3, Hz, 1H, HC(7)), 2.24 (dt, J=14.5, 8.0 Hz, 1H, HC(7)).

¹³C NMR: (151 MHz, D₂O) δ 134.5 (C(8)), 117.8 (C(9)), 72.2 (C(6)), 71.7 (C(5)), 70.0 (C(2)), 70.0 (C(3)), 69.5 (C(4)), 63.2 (C(1)), 37.0 (C(7)).

Q-NMR: 85% (caffeine, D₂O)

HRMS: Calcd. for C₉H₁₈O₆Na (M+Na)⁺: 245.1001. Found: 245.0993.

### Example 20. Representative Allylation Procedure C (AllylBF₃K, In Powder): (2S,3S,4R,5R)-2-Allylhexane-1,2,3,4,5,6-hexaol (12) from D-Fructose

The following synthetic scheme was followed:

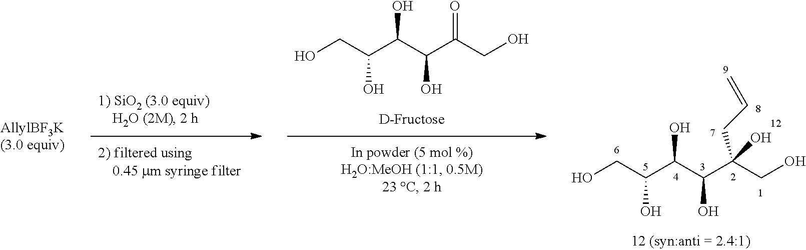

Following Representative Procedure C, a solution of D-fructose (180 mg, 1.00 mmol) with all other reaction components was stirred for 2 h whereupon the identical workup afforded 225 mg (93% purity (Q-NMR), 94% yield w.r.t purity) of (12) as a colorless glass.

Data for (12):

d.r.: 2.4:1 (C(2)/C(3); syn anti)

¹H NMR: (600 MHz, D₂O) δ 5.87 (ddt, J=17.4, 10.0, 7.3 Hz, 1H, HC(8)), 5.22-5.14 (m, 2H, HC(9)), 3.87 (s, 1H, HC(3)), 3.85-3.77 (m, 2H, HC(4, 6)), 3.72 (ddd, J=8.8, 6.3, 2.8 Hz, 1H, HC(5)), 3.67-3.58 (m, 3H, HC(1,6)), 2.49-2.32 (m, 2H, HC(7)).

¹³C NMR: (151 MHz, D₂O) δ 132.6 (C(8)), 119.0 (C(9)), 76.6 (C(2)), 71.2 (C(5)), 70.8 (C(3)), 69.6 (C(4)), 63.5 (C(1)), 62.9 (C(6)), 37.8 (C(7)).

Q-NMR: 93% (caffeine, D₂O)

HRMS: Calcd. for C₉H₁₈O₆Na (M+Na)⁺: 245.1001. Found: 245.0994.

### Example 21. Gram-Scale Allylation of D-Arabinose: (2R,3R,4R,5S)-Oct-7-ene-1,2,3,4,5-pentaol (9)—10 mmol Scale

The following synthetic scheme was followed:

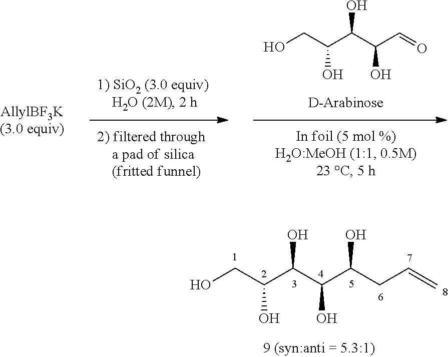

A 100 mL round-bottomed flask with a stir bar (3×1 cm) was charged with allyl-BF₃K (4.44 g, 30.0 mmol, 3 equiv) followed by silica gel (SiO₂, 1.80 g, 230-400 mesh, 30.0 mmol, 3.0 equiv) and then distilled H₂O (15 mL, 2.0 M). The suspension was vigorously stirred for 2 h whereupon the solution turned pale-yellow and the allylBF₃K dissolved. After the elapsed time, the solution was filtered through a pad of celite (1 cm×5 cm diameter) to obtain approximately 10 mL of clear pale-yellow filtrate (ca. 15 mmol, 1.5 equiv of allylboronic acid). A new 100 mL round-bottomed flask with a stir bar (3×1 cm) was charged with indium foil (0.127 mm, 58.3 mg, 5 mol %) and D-arabinose (1.50 g, 1.00 mmol) followed by simultaneous addition of the filtrate (10 mL, 1.0 M) and MeOH (10 mL, 1.0 M) and the suspension was stirred for 8 h at 23° C. The suspension was transferred to a 250 mL round-bottomed flask with successive rinsing of the vial with MeOH (100 mL). The solution was concentrated under reduced pressure (60° C., 15 mmHg) to afford a clear, colorless syrup. The syrup was dissolved in MeOH (50 mL) and filtered through a pad of silica (5 cm×5 cm diameter) and the filter cake was washed with MeOH (200 mL). The clear, colorless filtrate was concentrated under reduced pressure (60° C., 15 mmHg) to obtain a clear, colorless syrup. The syrup was dissolved in a minimal amount of MeOH (~15 mL), filtered through a syringe filter (0.22 μm), and the filtrate was concentrated under reduced pressure (60° C., 15 mmHg) to obtain a clear, colorless syrup. The syrup was dissolved in distilled H₂O (~1 mL) and filtered through a syringe filter (0.45 μm). The filtrate was concentrated under reduced pressure (60° C., 15 mmHg) to yield 1.92 g (94% purity (Q-NMR), 94% yield) of (9) as a white solid. All characterization data was identical as described above.

### Example 22. Glucose Allylation with AllylBpin in Flow

### Flow Reactor Catalyst Loading

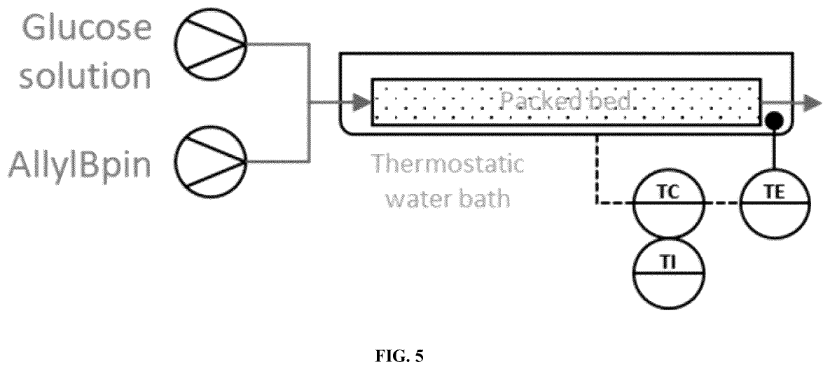

Initial design of the flow system involved the use of two syringe pumps. One pump contained a 1.6 M glucose solution in ethanol/water (1:1), while the other contained neat allylboronic acid pinacol ester (allylBpin), as illustrated in FIG. 5. Dimethyl sulfone was used as an internal standard for Q-NMR yield calculation and was dissolved in the glucose solution. A lab-scale tubular reactor was used for experiments that used stainless-steel tubing (4.45 mm ID, 78.5 mm length) and standard threaded connections for gas systems.

To select the catalyst loading in the bed, the stainless-steel reactor was packed with varying ratios of In(0) powder (100 mesh) and glass beads (100 μm), ranging from 1:0 to a 1:5 mass ratio of In(0) powder to glass beads. Experiments were conducted at different flow rates for both the glucose solution and allylBpin. At room temperature, the reactor afforded low yields (<10% product) at all analyzed flow rates. However, increasing the temperature improved the yield. The stoichiometry of allylBpin (2.00 equiv w.r.t. glucose) was maintained at a constant temperature (60° C. in a water bath) and constant solvent mixture (ethanol/water (1:1)).

The experimental campaign began with In(0) powder (3.87 g) without glass beads. Good yields were observed only for low flowrates, while at higher flowrates yield would largely drop. When the reactor was unloaded, the bed was not completely wet and hollow regions were found, suggesting that In(0) powder would favor flow bypasses by partial compression of the powder; powder compression was also deemed to be the reason for the clogging of the reactor. The use of glass beads helped provide structural integrity to the catalytic bed (no hollow regions were found when unloading the reactor), while also achieving better efficiency with respect to the use of In(0) powder on a wider set of flowrates the performance dropped only at very high flowrates. This effect likely obtains because the glass beads act as passive mixers and do not allow the In(0) particles to agglomerate and clog the reactor.

Individual flowrates are computed as follows:

$${\dot{Q}}_{g l u} = \frac{\dot{Q}}{1 + R} , {\dot{Q}}_{B p i n} = \frac{R \dot{Q}}{1 + R} , R = \frac{b {c}_{g l u}^{s t o c k}}{{c}_{B p i n}^{n e a t}} = 0.6$$

\dot{Q}_{glu}=flow rate of glucose, \dot{Q}=total flow rate, \dot{Q}_{Bpin}=flow rate of allylBpin, b=allylBpin (equiv), c=concentration.

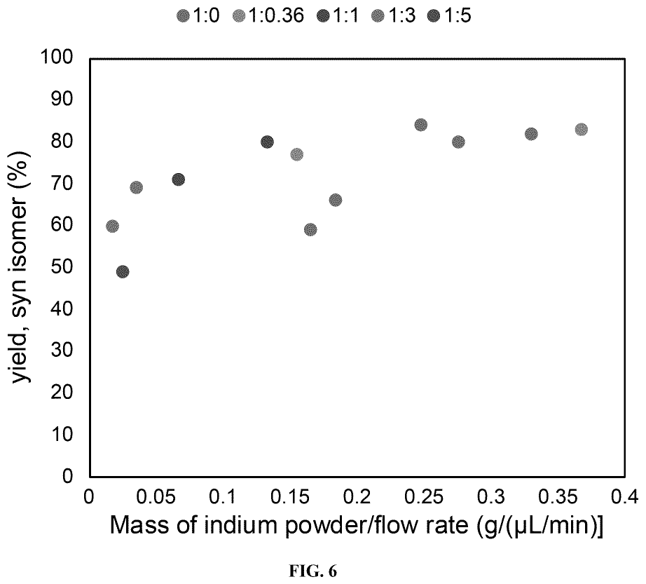

Analysis of the results (FIG. 6) suggested that the 1:3 In(0) powder/glass ratio was the proper balance between satisfactory yield (to be optimized in the subsequent Bayesian optimization) and In(0) use. A blank test was also run with no indium (only glass beads) and no conversion was observed.

### Bayesian Optimization

Reaction optimization can be performed adopting different techniques, with experimental campaigns driven by user experience or phenomenological models, by more guided Design of Experiment (DoE), or by Artificial Intelligence, such as Bayesian Optimization (BO) and Neural-Network (NN) based methods. The optimization variables can affect the reaction outcomes in manners that are difficult to grasp initially by a practitioner, thus non-guided optimization methods can lead to biases or non-optimal solutions. When the number of variables is large, visualization of the outcomes becomes complex and DoE methods may require an excessive number of experiments to confidently lead to optimal conditions. A NN on the other hand can provide good suggestions especially when different reaction substrates are considered, yet large databases (with both experimental and computational data) are needed to train NNs. This requirement may become an issue when the substrate is defined, and no data is available for similar reactions or when transport phenomena can affect the reactivity. A good balance can be achieved with BO, able to deal with many variables and requiring smaller datasets. This technique leverages Gaussian Processes (GP) to describe data (with a sufficiently flexible fitting object) and suggest new conditions to test to reach optimality within a limited number of experiments. These methods are able to predict the outcomes of a reaction and provide an uncertainty of such prediction.

The reaction considered in this work is new, in its chemistry and its practical application, because of the use of elemental indium as a catalytic bed in a flow system. In this work, BO was employed to take advantage of its ability to consider many variables with complex interdependencies (chemistry and potential transport phenomena, which could be hardly described through a complete phenomenological model) while requiring a limited amount of data to start guiding the user throughout optimization, overcoming the drawbacks of both DoE and NN methods for this specific application.

### Design Space

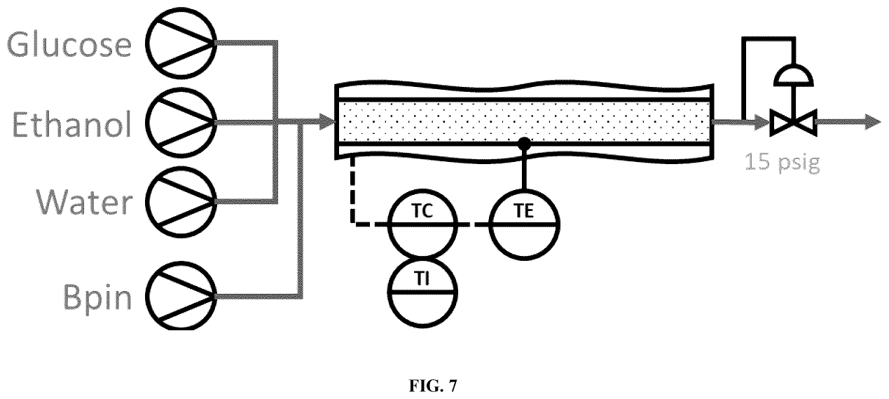

The same lab-scale tubular reactor used in the preliminary experiments (4.45 mm ID, 78.5 mm length) packed with 1:3 In(0) powder/glass ratio and standard threaded connections for gas systems was employed. Four fluids (glucose (5.00 M solution in water), allylBpin (neat), water, and ethanol) were fed into the reactor using PHD ULTRA™ syringe pumps (FIG. 8) from Harvard Apparatus and PTFE tubing ( 1/32″ ID). The 5.00 M glucose solution also contained dimethyl sulfone as an internal standard for Q-NMR. During all experiments, the reactor was kept at set temperatures using thermal tape (on external wall of the tube) and a PID feedback controller with a K-type thermocouple (FIG. 7 and FIG. 8). Pressure was set at the outlet of the steel tube using a Back-Pressure Regulator set at 15 psig.

The design space was constructed as a four-dimensional domain:
Total flowrate, \dot{Q}: 17.5-52.5 μL/min;
Temperature, T: 50-80° C.;
Equivalents of allylboronic pinacol ester (w.r.t. glucose), b: 1.0-2.5;
Ethanol fraction in the inlet glucose solution, x_{EtOH}: 30-50%.

Points of the design space are represented as 4-dimensional vectors X=(\dot{Q}, T, b, x_{EtOH}). The domain boundaries were set according to the extreme conditions that the experimental setup could handle. In particular, the equivalents of allylBpin were selected from stoichiometric (1.0) to a large excess (2.5). The ethanol volume fraction was set by solubility tests on glucose solutions. Above 50% ethanol, glucose would precipitate, whereas below 30% ethanol, pinacol (the only byproduct of the reaction) would precipitate. Both conditions would cause clogging of the reactor and the downstream lines. The vapor pressure of the solvent (estimated via Raoult's law for simplicity) imposed the minimum value for the backpressure regulator setpoint to avoid boiling inside the reactor. Given the flowrates explored (which roughly correspond to contact times ranging from 10 to 30 min), the pumps were not able to operate above 15 psig backpressure, thus imposing a maximum temperature (by reverting Raoult's law) of 80° C. The pumps were not able to operate at higher backpressures as the reactor packing is made of fine particles and the viscosity of the reacting stream increases throughout the reaction, thus causing high pressure drops over the catalytic bed.

### Example 23. Analysis of the Indium Content of Millad NX 8000 Intermediates and Products (Batch)

The following synthetic scheme was followed:

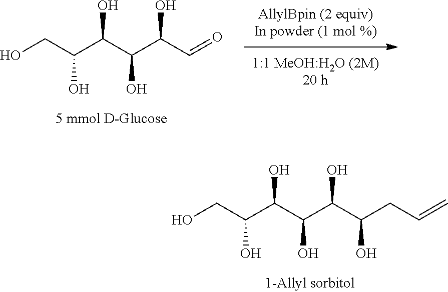

The results of the analysis are presented in Table 1.

TABLE 1

| | Filtered Reaction | | Purified | | | Purified |
|---|---|---|---|---|---|---|
| | Solution | Indium | Allylsorbitol | Indium | | Allylsorbitol |
| | ICP-MS Sample | Concentration | ICP-MS Sample | Concentration | | Yield |
| Reaction A | MA-1A | 381 ppm | MA-2A | 151 | ppm | 1.2663 g |
| Reaction B | MA-1B | 324 ppm | MA-2B | 80 | ppm | 1.2908 g |
| Reaction C | MA-1C | 356 ppm | MA-2C | 1363 | ppm | 1.2580 g |

Indium powder (5.7 mg, 0.05 mmol) and D-glucose (180 mg, 5 mmol) were added to a 7 mL glass vial along with a ⅜″× 3/16″ egg-shaped magnetic stir bar. 1.25 mL of distilled water was added, and the mixture was stirred with gentle heating until the glucose was fully dissolved and allowed to cool. 1.25 mL of methanol was added followed by 1.88 mL of allylboronic acid pinacol ester (1.68 g, 10 mmol). The reaction was stirred at 1000 rpm for 20 hours. Full conversion was confirmed by ¹H NMR.

Note: The following procedure details an alternative purification method that avoids the use of dichloromethane and lyophilization.

The reaction mixture was filtered through a 0.22 μm syringe filter to remove the indium, and a 100 μL sample of the filtrate was diluted with 5 mL ultrapure concentrated nitric acid and submitted for ICP-MS analysis (samples MA-1X). The reaction vial and filter were washed with 5 mL of methanol which was combined with the filtrate. This clear, colorless solution was concentrated with a rotary evaporator (60° C., 200-20 Torr) yielding a clear, colorless syrup. The syrup was dissolved in 250 mL of pH 3 methanol containing 10⁻³ M HCl and again concentrated to a syrup (60° C., 200-20 Torr). This step was repeated an additional three times to completely remove the boric acid byproduct as confirmed by ¹¹B NMR. The syrup was then dissolved in 200 mL of distilled water and concentrated to a syrup (60° C., 100-20 Torr). This step was repeated an additional three times to completely remove the pinacol byproduct as confirmed by ¹H NMR. The allylsorbitol product was obtained as a clear, colorless syrup which crystallized upon standing (1.2663 g, 1.2908 g, 1.2580 g for triplicate reactions). The product diastereomeric ratio was found to be 7:1 favoring the syn isomer. ~20 mg samples of the purified product were submitted for ICP-MS analysis (samples MA-2X). The indium concentrations were found to range from 80-1363 ppm. This large range was unexpected given the relatively consistent concentrations found in the previous samples. It is believed that the low concentrations found in samples MA-2A and MA-2B are erroneous, possibly due to nonuniform distribution of indium in the purified allylsorbitol. This is supported by the high concentration found in sample MA-3B, which consists of a hydrogenated solution of the material from which MA-2B was sampled.

The following synthetic scheme was followed for the product of the previous reaction:

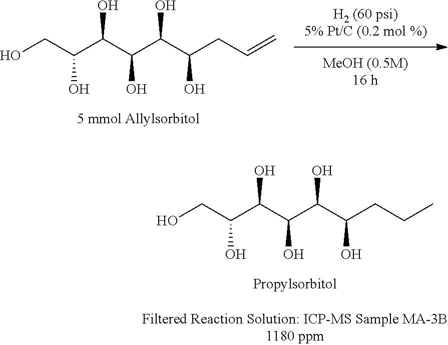

This reaction was first attempted using the purified product obtained from allylation Reaction A using ethanol as the solvent. During the reaction, the propylsorbitol product precipitated resulting in the reaction mixture solidifying and incomplete conversion observed by

¹H NMR. The reaction was repeated as described below with methanol as the solvent and using the purified product obtained from allylation Reaction B, and no precipitation was observed.

Allylsorbitol (1.29 g, 5 mmol) used directly as obtained from the previously described method (Reaction B) was dissolved in 10 mL of anhydrous methanol and added to a 20 mL glass vial along with 37 mg of platinum on carbon (5 wt. % loading) and a ⅝″×¼″ egg-shaped magnetic stir bar. The vial was loaded into a steel reactor which was pressurized to 60 μsi with hydrogen and then stirred at 600 rpm for 16 hours. Full conversion was confirmed by ¹H NMR.

The reaction mixture was filtered through 0.5 cm of celite in a glass Pasteur pipette plugged with glass wool to remove the catalyst. A 100 μL sample of the clear, colorless filtrate was submitted for ICP-MS analysis (sample MA-3B). The reaction vial and celite were washed with 10 mL of methanol which was combined with the filtrate.

The following synthetic scheme was followed, using the product of the previous reaction as starting material:

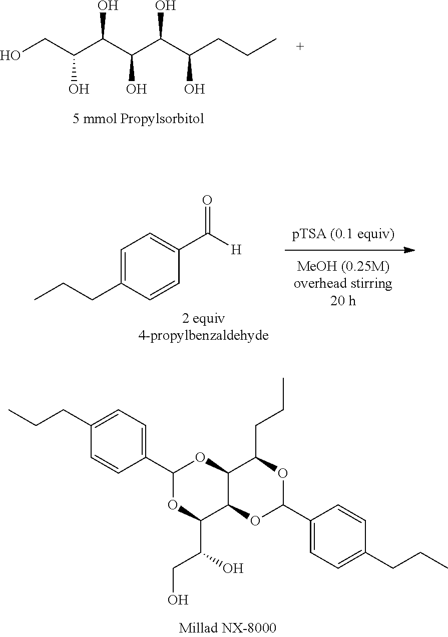

The results of the analysis are presented in Table 2.

TABLE 2

| | ICP-MS | | |
|---|---|---|---|
| | Sample | Indium | |
| Sample Source | Name | Concentration | |
| Reaction solution after product filtered off | MA-4B | 1224 | ppm |
| Solid product after washing with methanol | MA-5B | 106 | ppm |
| Solid product after washing with water | MA-6B | 30 | ppm* |
| Pure product after washing with boiling | Millad-pure | 16 | ppm* |
| water and drying | | | |

*Measured intensity is close to the measured intensity of the blank.

The solution of propylsorbitol in methanol (20 mL, 5 mmol) used directly as obtained from the previously described reaction was added to a 25 mL round-bottom flask followed by p-toluenesulfonic acid monohydrate (95 mg, 0.5 mmol) and 4-propylbenzaldehyde (1.48 g, 10 mmol). Using an overhead mechanical stirrer with a 3.3 cm×1.2 cm paddle, the reaction was stirred at 900 rpm for 20 hours during which time the insoluble product formed a thick, white slurry.

The slurry of product was vacuum filtered through a paper filter, and a sample of this filtrate was submitted for ICP-MS analysis (sample MA-4B). The reaction flask and filter cake were washed twice with 10 mL of methanol, and the product was allowed to partially dry on the filter. A 20 mg sample of the product was dried under high vacuum and submitted for ICP-MS analysis (sample MA-5B). The damp, light-grey solid was transferred to a 20 mL glass vial and dispersed in 15 mL of distilled water using a steel spatula to break up the insoluble mass of product. The resulting slurry was again vacuum filtered through a paper filter, and the vial and filter cake were washed with another 10 mL of distilled water. The filtrate was tested using pH paper and found to be acidic (pH 3). This distilled water wash was therefore repeated, and the resulting filtrate was found to be nearly neutral (pH 6). A 20 mg sample of the product was dried under high vacuum and submitted for ICP-MS analysis (sample MA-6B).

The wet, light-grey solid was transferred to a 25 mL Erlenmeyer flask along with a ¾″×⅜″ egg-shaped magnetic stir bar and 20 mL of distilled water. The suspension was heated to boiling while stirring at 1500 rpm. The product fully dispersed in the boiling water forming a thick, white slurry of even consistency. The hot slurry was vacuum filtered through a paper filter, and the boiling flask and filter cake were washed twice with 10 mL of boiling distilled water. The product was then allowed to dry on the filter until cool. ¹H NMR analysis confirmed the product to be a single diastereomer of the desired bis-acetal, Millad NX-8000, with the only detectable impurities being water and a trace amount of 4-propylbenzaldehyde. The damp, light-grey solid was transferred to a glass dish and dried in a vacuum oven (100° C., 20 Torr) for 16 hours. ¹H NMR analysis confirmed the complete removal of the water and 4-propylbenzaldehyde impurities. The final product was obtained as a dry, white solid (1.59 g, 66% yield over 3 steps) and submitted for ICP-MS analysis (sample Millad-pure).

### Example 24. Analysis of the Indium Content of Allyl Sorbitol (Flow)

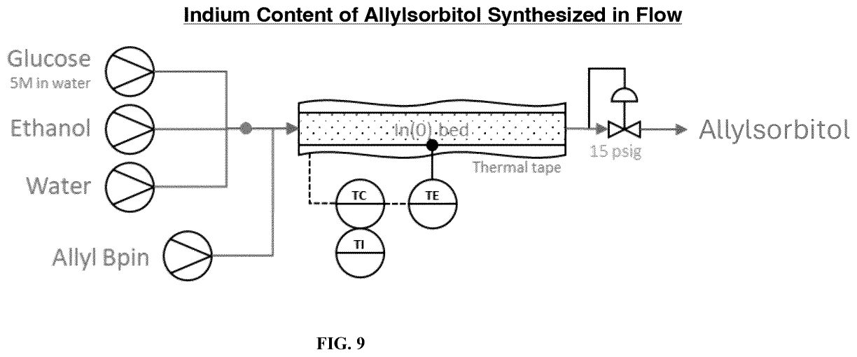

The analysis of the indium content of allyl sorbitol synthesized in flow was done on samples of allyl sorbitol synthesized in an apparatus as shown in FIG. 9.

The allylsorbitol synthesis in flow uses a packed bed reactor consisting of a stainless-steel tube (0.175″ ID, 78.5 mm length) containing the catalyst bed material. This catalyst bed material consists of 0.61 g of 100 mesh indium powder evenly dispersed with 1.84 g of 100 μm glass beads. Four fluids glucose (5.00 M solution in water), neat allylBpin, water, and ethanol-were pumped into the heated reactor via PHD ULTRA syringe pumps. The flow rate of each of these solutions as well as the reactor temperature were optimized toward maximizing the reaction percent yield with respect to glucose (Conditions A) and, separately, the reaction productivity measured as the rate of desired product output (Conditions B). Table 3 lists the reaction parameters corresponding to these two sets of conditions as well as their resultant yields and productivities.

TABLE 3

| | Conditions A | Conditions B |
|---|---|---|
| | (Optimal | (Optimal |
| Reaction Parameter | Percent Yield) | Productivity) |
| 5M Glucose Flow Rate (μL/min) | 4.028 | 17.871 |
| Water Flow Rate (μL/min) | 0.824 | 0 |
| Ethanol Flow Rate (μL/min) | 3.204 | 17.871 |
| AllylBpin Flow Rate (μL/min) | 9.443 | 16.758 |
| Reactor Temperature | 80 °C | 80 °C |
| Measured Yield | 100% | 69% |
| Measured Productivity (g/hr) | 0.239 | 0.735 |

The solution obtained using Conditions A could be purified using the standard purification procedure to obtain allylsorbitol in its pure form. With the system operating under Conditions A for 5 hours, approximately 5.25 mL of crude product solution was collected and diluted with 50 mL of methanol. This solution was then concentrated under reduced pressure (55-60° C., 15 mmHg). The viscous residue was resubjected to this dilution and evaporation protocol six times to yield a semi-solid residue free of the boric acid byproduct. The residue was then dissolved in 15 mL of water and lyophilized at 0.2 mmHg for 4 days yielding a white solid. This solid was sonicated in 20 mL of dichloromethane for 3 minutes and then collected by filtration using a fritted funnel to afford allylsorbitol as a white solid free of the pinacol byproduct. ICP-MS analysis of this purified product found an indium concentration of 277 ppm.

The crude product solution obtained using Conditions B was analyzed directly and found to have an indium concentration of 76 ppm.

All publications mentioned herein are incorporated by reference to the extent they support the present invention.

### REFERENCES

A number of patents and publications are cited above in order to more fully describe and disclose the invention and the state of the art to which the invention pertains. Full citations for these references are provided below. Each of these references is incorporated herein by reference in its entirety into the present disclosure, to the same extent as if each individual reference was specifically and individually indicated to be incorporated by reference.
[1]. Hernindez-Fernindez, J., Puello-Polo, E. & Marquez, E. Identifying, Quantifying, and Recovering a Sorbitol-Type Petrochemical Additive in Industrial Wastewater and Its Subsequent Application in a Polymeric Matrix as a Nucleating Agent. Molecules 28, 4948 (2023).
[2]. Milliken invests to meet global demand for PP clarifier. Addit. Polym. 2014, 7, doi: https://doi.org/10.1016/S0306-3747(14)70090-9 (2014).
[3]. Zhou, F. & Li, C.-J. En route to metal-mediated and metal-catalysed reactions in water. Chem. Sci. 10, 34-46 (2019).
[4]. Hanessian, S. Total Synthesis of Natural Products: The ‘Chiron’ Approach, Pergamon Press: Oxford, 1983.
[5]. Hanessian, S., Giroux, S., Merner, B. L. Design and Strategy in Organic Synthesis, Wiley-VCH: Weinheim, 2013.
[6]. Newhouse, T., Baran, P. S. & Hoffmann, R. W. The Economies of Synthesis. Chem. Soc. Rev. 38, 3010-3021 (2009).
[7]. Trost, B. M. The atom economy—a search for synthetic efficiency. Science 254, 1471-1477 (1991).
[8]. Wender, P. A., Verma, V. A., Paxton, T. J. & Pillow, T. H. Function-oriented synthesis, step economy, and drug design. Acc. Chem. Res. 41, 40-49 (2008).
[9]. Chung, K. & Waymouth, R. M. Selective Catalytic Oxidation of Unprotected Carbohydrates. ACS Catal. 6, 4653-4659, (2016).
[10]. Kan, J. et al. Umpolung carbonyls enable direct allylation and olefination of carbohydrates. Sci. Adv. 8, eabm6840 (2022).
[11]. Schmid, W. & Whitesides, G. M. Carbon-carbon bond formation in aqueous ethanol: diastereoselective transformation of unprotected carbohydrates to higher carbon sugars using allyl bromide and tin metal. J. Am. Chem. Soc. 113, 6674-6675 (1991).
[12]. Kim, E., Gordon, D. M., Schmid, W. & Whitesides, G. M. Tin- and indium-mediated allylation in aqueous media: application to unprotected carbohydrates. J. Org. Chem. 58, 5500-5507 (1993).
[13]. Chan, T.-H. & Li, C.-J. A concise chemical synthesis of (+)-3-deoxy-D-glycero-D-galacto-nonulosonic acid (KDN). J. Chem. Soc., Chem. Commun., 747-748 (1992).
[14]. Petrier, C. & Luche, J. L. Allylzinc reagents additions in aqueous media. J. Org. Chem. 50, 910-912, (1985).
[15]. Nokami, J., Otera, J., Sudo, T. & Okawara, R. Allylation of aldehydes and ketones in the presence of water by allylic bromides, metallic tin, and aluminum. Organometallics 2, 191-193 (1983).
[16]. Tan, K.-T., Chang, S.-S., Cheng, H.-S. & Loh, T.-P. Development of a Highly α-Regioselective Metal-Mediated Allylation Reaction in Aqueous Media: New Mechanistic Proposal for the Origin of α-Homoallylic Alcohols. J. Am. Chem. Soc. 125, 2958-2963, (2003).
[17]. Kumar, D., Vemula, S. R., Balasubramanian, N. & Cook, G. R. Indium-Mediated Stereoselective Allylation. Acc. Chem. Res. 49, 2169-2178, (2016).
[18]. Xie, C. Dibenzylidene Sorbitol (DBS)-Based Compounds, Compositionss and Methods for Using Such Compounds US 2007/0249850 A1, Oct. 25, 2007.
[19]. Xie, C. Substituted Alditol Compounds, Compositions, and Methods. U.S. Pat. No. 7,888,454 B2, Feb. 15, 2011.
[20]. AgileIntel Research (ChenIntel360). (Jul. 5, 2023) “Market Volume of Polypropylene Worldwide from 2015 to 2022, with a Forecast for 2023 to 2030 (in Million Metric Tons).,” can be found under https://www.statista.com/statistics/1245169/polypropylene-market-volume-worldwide, (accessed: February 2024).
[21]. Tullo, A. H., Making Clearer Polypropylene. Chem. Eng. News 2010, 88 (36), 34-36.
[22]. Yus, M., Gonzalez-Gomez, J. C. & Foubelo, F. Catalytic enantioselective allylation of carbonyl compounds and imines. Chem. Rev. 111, 7774-7854 (2011).
[23]. Mlynarski, S. N., Schuster, C. H. & Morken, J. P. Asymmetric synthesis from terminal alkenes by cascades of diboration and cross-coupling. Nature 505, 386-390 (2014).
[24]. Denmark, S. E.; Fu, J., Catalytic Enantioselective Addition of Allylic Organometallic Reagents to Aldehydes and Ketones. Chem. Rev. 103 (8), 2763-2794 (2003).
[25]. Schneider, U., Ueno, M. & Kobayashi, S. Catalytic use of indium (0) for carbon-carbon bond transformations in water: general catalytic allylations of ketones with allylboronates. J. Am. Chem. Soc. 130, 13824-13825 (2008).
[26]. AllylBPin—$269/mol—AK Scientific; AllylBF₃K—$286/mol—Aaronchem; AllylBr—$30/mol—Sigma.
[27]. Molander, G. A.; Cavalcanti, L. N.; Canturk, B.; Pan, P. S.; Kennedy, L. E. Efficient Hydrolysis of Organotrifluoroborates via Silica Gel and Water. J. Org. Chem., 74 (19), 7364-7369 (2009).
[28]. Paquette, L. A.; Mitzel, T. M. Addition of Allylindium Reagents to Aldehydes Substituted at Cα or Cβ with Heteroatomic Functional Groups. Analysis of the Modulation in Diastereoselectivity Attainable in Aqueous, Organic, and Mixed Solvent Systems. J. Am. Chem. Soc. 118 (8), 1931-1937 (1996).
[29]. Gordon, D. M. & Whitesides, G. M. Indium-mediated allylations of unprotected carbohydrates in aqueous media: a short synthesis of sialic acid. J. Org. Chem. 58, 7937-7938 (1993).
[30]. Coley, C. W. et al. A robotic platform for flow synthesis of organic compounds informed by AI planning. Science 365, eaax1566 (2019).
[31]. Nambiar, A. M. K. et al., Bayesian optimization of computer-proposed multistep synthetic routes on an automated robotic flow platform, ACS Cent. Sci., 8, (6) 825-836 (2022).
[32]. Shields, B. J. et al. Bayesian reaction optimization as a tool for chemical synthesis. Nature 590, 89-96 (2021).
[33]. Otte, D. A. L., Borchmann, D. E., Lin, C., Weck, M. & Woerpel, K. A. 13C NMR Spectroscopy for the Quantitative Determination of Compound Ratios and Polymer End Groups. Org. Lett. 16, 1566-1569, doi:10.1021/o1403776k (2014).
[34]. Wang, Y., Carder, H. M. & Wendlandt, A. E. Synthesis of rare sugar isomers through site-selective epimerization. Nature 578, 403-408, doi:10.1038/s41586-020-1937-1 (2020).

We claim:

1. A composition comprising one or more compounds chosen from allyl sorbitol, allenyl sorbitol, propargyl sorbitol, propyl sorbitol, and ((R)-1-((2R,4R,4aS,6R,8R,8aS)-8-propyl-2,6-bis(4-propylphenyl)tetrahydro-[1,3]dioxino[5,4-d][1,3]dioxin-4-yl)ethane-1,2-diol) and indium metal and/or an indium salt.

2. The composition of claim 1, wherein said indium salt is chosen from InCl, InI, InBr, InCl₃, InBr₃, InI₃, In(OAc)₃, In(OTf)₃In₂O₃, or In(OH)₃, and combinations thereof.

3. The composition of claim 1, wherein said composition comprises allyl sorbitol, and indium metal and/or an indium salt.

4. The composition of claim 1, wherein said indium metal and/or indium salt is present at a concentration of about 16 ppm to about 300 ppm.

5. The composition of claim 1, wherein said composition does not include compounds of Formulae II, IIA, IIB, IID, and/or allylBF₃K: 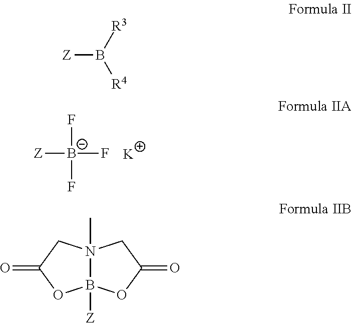
wherein R³ and R⁴ are independently chosen from H, hydroxy, halogen, and C₁₋₆ alkyl or combinations thereof;
or R³ and R⁴, taken together with the B atom to which they are attached, form a 5-membered, a 6-membered, or a 7-membered heterocyclic ring system; and
Z is 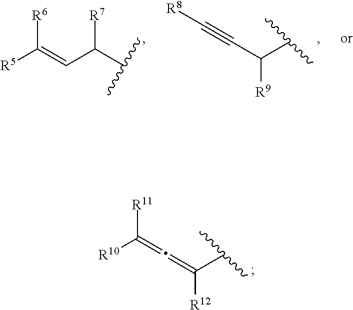
and
wherein R⁵-R¹² are independently chosen from H, hydroxy, halogen, and C₁₋₆ alkyl or a combination thereof.

6. A method of preparing a compound of Formula I, said method comprising reacting a monosaccharide and a compound of any of Formulae II, IIA, or IIB in the presence of indium metal and/or an indium salt and at least one solvent 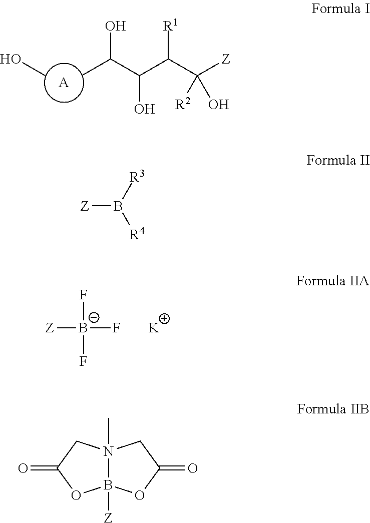
wherein:
A is chosen from —CH₂—, 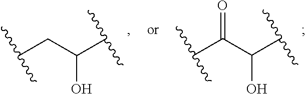 [SMILES: *CC(*)O ; *C(=O)C(*)O]
R¹ is H, —OH, or 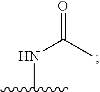 [SMILES: *NC(C)=O]
R² is H or —CH₂OH;
R³ and R⁴ are independently chosen from hydroxy, halogen, and C₁₋₆ alkoxy or combinations thereof, or R³ and R⁴, taken together with the B atom to which they are attached, form a 5-membered, a 6-membered, or a 7-membered heterocyclic ring system; and
Z is 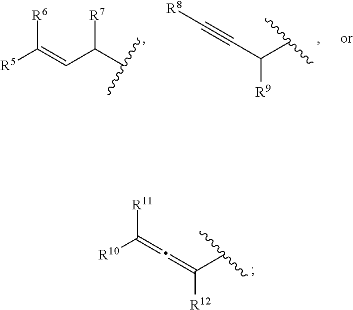
wherein R⁵-R¹² are independently chosen from H, hydroxy, halogen, and C₁₋₆ alkyl or a combination thereof.

7. The method of claim 6, wherein said monosaccharide is chosen from glucose, D-glucose, hexose, pentose, and combinations thereof.

8. The method of claim 6, wherein said at least one solvent is chosen from water, methanol, ethanol, isopropanol, acetonitrile, tetrahydrofuran, and dimethylformamide, and or a combination thereof.

9. The method of claim 6, wherein said compounds of Formula IA and/or Formula IB are prepared by reacting a monosaccharide of Formula III and/or stereoisomers thereof, and a compound of Formula IIC, in the presence of indium metal and/or an indium salt

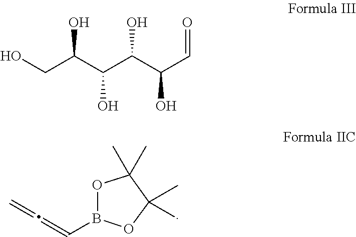

10. The method of claim 6, wherein said compounds of Formula IA and/or IB are reacted with hydrogen in the presence of a catalyst to obtain a compound of Formula IV, or a stereoisomer thereof:

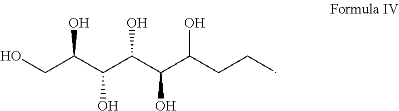

11. The method of claim 6, with the proviso that when said monosaccharide is glucose, then for Formula II, if Z is 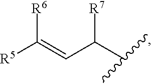
and R⁵, R⁶ and R⁷ are independently H, then R³ and R⁴ taken together is not: 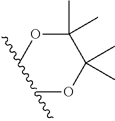
and does not form a compound of Formula IID: 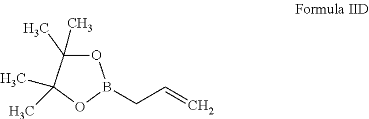
or with the proviso that when said monosaccharide is glucose, then for Formula IIA, Z is not 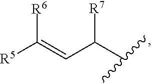
or Formula IIA is not allylBF₃K, or Formula II is not allyl boronic acid pinacol ester (Formula IID).

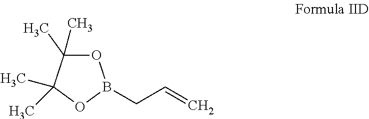

12. A continuous process of preparing a compound of Formula I, said method comprising reacting a monosaccharide (“reactant 1”) and a compound of any of Formulae II, IIA, or IIB (“reactant 2”) in the presence of indium metal and/or an indium salt and at least one solvent, 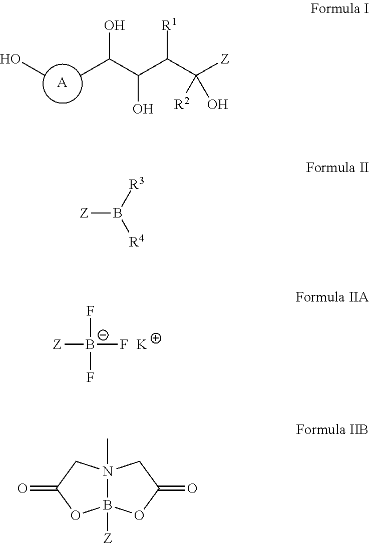
wherein:
A is chosen from CH₂, 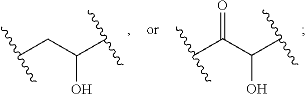 [SMILES: *CC(*)O ; *C(=O)C(*)O]
R¹ is H, —OH, or 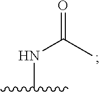 [SMILES: *NC(C)=O]
and
R² is H or —CH₂OH;
R³ and R⁴ are independently chosen from H, hydroxy, halogen, and C₁₋₆ alkoxy or combinations thereof, or
R³ and R⁴, taken together with the B atom to which they are attached, form a 5-membered, a 6-membered, or a 7-membered heterocyclic ring system; and
Z is 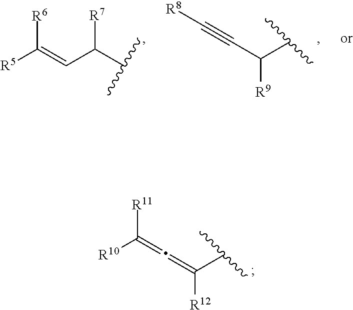
wherein R⁵-R¹² are independently chosen from H, hydroxy, halogen, and C₁₋₆ alkyl or combinations thereof,
wherein said reactants and solvent are introduced into a reactor via separate;
wherein said indium metal and/or an indium salt is arranged to form a catalytic bed,
optionally wherein said reaction is heated;
optionally wherein said indium metal and/or an indium salt is reused for more than one reaction;
optionally wherein said indium metal and/or an indium salt is mixed with an inert material such as glass beads, ceramic beads, or metallic beads.

13. The method of claim 12, wherein said reactants and solvent are independently introduced into the reactor at a flow rate in the range of about 15 μL/min to about 55 μL/min.

14. The method of claim 12, wherein said reaction is heated to a temperature in the range of about 20° C. (e.g., room temperature) to about 100° C.

15. The method of claim 12, wherein said compound of Formula I is one of: 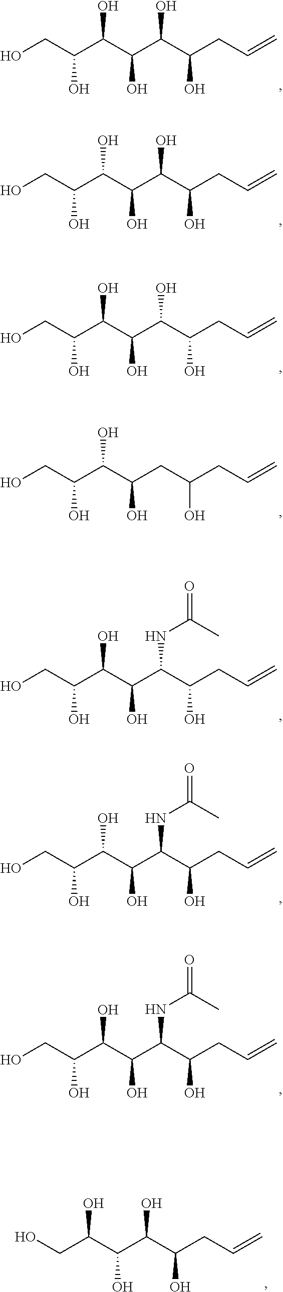 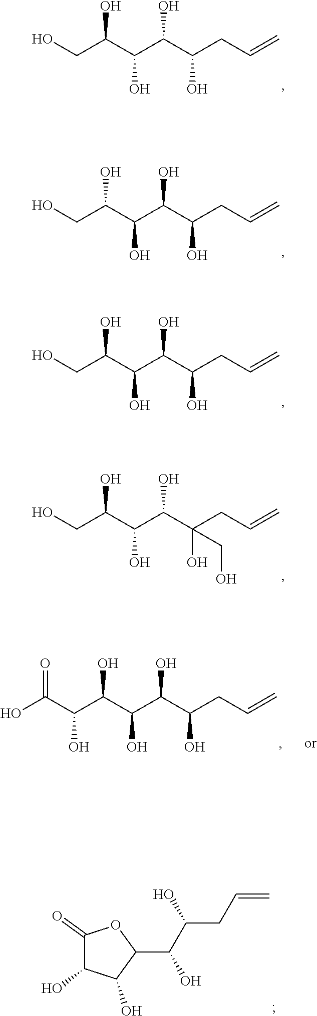 [SMILES: C=CC[C@H](O)[C@@H](O)[C@H](O)[C@H](O)CO ; C=CC[C@@H](O)[C@H](O)[C@@H](O)[C@@H](O)CO ; C=CC[C@@H](O)[C@H](O)[C@@H](O)[C@H](O)CO ; C=CCC(O)(CO)[C@@H](O)[C@H](O)[C@H](O)CO ; C=CC[C@@H](O)[C@H](O)[C@@H](O)[C@H](O)[C@H](O)C(=O)O ; C=CC[C@@H](O)[C@H](O)C1OC(=O)[C@@H](O)[C@H]1O]
and
wherein said monosaccharide is chosen from D-glucose, D-galactose, D-mannose, 2-deoxy-D-galactose, N—Ac-D-mannosamine, N—Ac-D-galactosamine, N—Ac-D-glucosamine, D-ribose, D-arabinose, L-arabinose, D-xylose, D-fructose, D-glucuronolactone, and D-glucoronic acid.

16. The method of claim 12, wherein R³ and R⁴, taken together with the B atom to which they are attached, form a 5-membered, a 6-membered, or a 7-membered heterocyclic ring system with at least two oxygen or nitrogen atoms (e.g., two oxygen atoms connect directly to the boron atom).

17. The method of claim 12, wherein said indium salt is chosen from InCl₃, InBr₃, InI₃, In(OTf)₃, In(OAc)₃, InI, InBr, InCl, In₂O₃, or In(OH)₃, or combinations thereof.

18. The method of claim 12, wherein said at least one solvent is chosen from water, methanol, ethanol, isopropanol, acetonitrile, tetrahydrofuran, and dimethylformamide, and or a combination thereof.
